(19) United States
(12) Patent Application Publication
(10) Pub. No.: US 2025/0228824 A1
(43) Pub. Date: Jul. 17, 2025

(54) COMPOSITIONS AND METHODS FOR INHIBITION OF ITO AS TREATMENT FOR THE J WAVE SYNDROMES AND HYPOTHERMIA

(71) Applicants: Lankenau Institute for Medical Research, Wynnewood, PA (US); Simon Fraser University, Burnaby (CA)

(72) Inventors: Charles Antzelevitch, Norton, MA (US); Mena Fawzy Abdelsayed, Port Coquitlam (CA); Peter Charles Ruben, Anmore (CA)

(21) Appl. No.: 18/697,657

(22) PCT Filed: Oct. 3, 2022
(86) PCT No.: PCT/US2022/077474

§ 371 Date: Apr. 1, 2024

Related U.S. Application Data
(60) Provisional application No. 63/251,176, filed on Oct. 1, 2021.

(51) Int. Cl. A61K 31/427; A61K 31/137; A61K 31/40; A61K 31/4184; A61K 31/444; A61K 31/4709; A61K 31/498; A61P 9/00

(52) U.S. Cl. CPC A61K 31/427; A61K 31/137; A61K 31/40; A61K 31/4184; A61K 31/444; A61K 31/4709; A61K 31/498; A61P 9/00

(57) ABSTRACT

Compositions and methods for treating J Wave Syndromes via ion-channel specific and cardio-selective inhibition of Ito are disclosed. In certain embodiments, the method includes administering a composition comprising ARumenamide-787 (AR-787) to a subject.

### CROSS REFERENCE TO RELATED PATENT APPLICATIONS

This patent application claims the benefit of U.S. Provisional Patent Application No. 63/251,176, filed Oct. 1, 2021. The entire contents of the foregoing application are incorporated herein by reference, including all text, tables, drawings, and sequences.

### GRANT SUPPORT STATEMENT

This invention was made with government support under Grant Numbers HL47678, HL138103, and HL152201 awarded by National Institute of Health. The government has certain rights in the invention.

### FIELD OF THE INVENTION

This invention relates to the fields of cardiac disease and means for efficient treatment via adminstration of ion-channel specific and cardio-selective inhibitors of Iₜₒ, which plays a major role in J Wave Syndromes.

### BACKGROUND OF THE INVENTION

The J wave syndromes (JWS) are characterized by distinctive J waves and/or ST segment elevation in specific ECG-leads and are associated with risk for development of polymorphic ventricular tachycardia and fibrillation (pVT and VF) leading to sudden cardiac death (SCD). The two principal forms of JWS are Brugada syndrome (BrS) and early repolarization syndrome (ERS).¹

First line treatment for high-risk patients suffering from JWS is an implantable cardioverter defibrillator (ICD), an approach that is problematic in young infants for whom a pharmacologic alternative would be preferable. Pharmacological therapy is also desirable for individuals experiencing frequent appropriate ICD shocks.² Thus, there is a need for safe and effective pharmacologic treatments capable of preventing life-threatening arrhythmic events associated with JWS.

### SUMMARY OF THE INVENTION

The present invention comprises methods for treating a J Wave Syndrome (JWS) in a patient in need thereof, comprising administering an effective amount of at least one I_{TO} inhibitor and/or I_{NA} augmentor. In certain embodiments the JWS is Brugada syndrome (BrS) or early repolarization syndrome (ERS). In certain embodiments, the treatment is effective at suppressing the electrocardiogramd/or arrhythmic manifestations of JWS. In another embodiment, treatment reduces instances of ventricular fibrillation and/or sudden cardiac death and/or the amplitude of J waves compared to an untreated control.

In another aspect, the at least one I_{TO} inhibitor and/or I_{NA} augmentor is selected from Table 1. In another aspect, the at least one I_{TO} inhibitor and/or I_{NA} augmentor is AR-787. In certain embodiments, the at least one I_{TO} inhibitor and/or I_{NA} augmentor is administered via enteral, parenteral, topical, or pulmonary administration.

In still another aspect, the treatment further comprises administration of at least one of an I_{CA} augmentor and/or at least one β-adrenergic agonist. In certain embodiments, the at least one ICA augmentor is isoproterenol, cilostazol and/or milrinone. In certain embodiments, the treatment further comprises administration of at least one of quinidine, bepridil, and denopamine.

In another aspect, the patient has hypothermia. In certain embodiments, the hypothermia is mild hypothermia, moderate hypothermia, severe hypothermia, or therapeutic hypothermia.

In another aspect, the present invention comprises methods for ameliorating symptoms of a J Wave Syndrome (JWS) phenotype in a patient having hypothermia, comprising administering an effective amount of at least one I_{TO} inhibitor and/or I_{NA} augmentor. In certain embodiments, the hypothermia is mild hypothermia, moderate hypothermia, severe hypothermia, or therapeutic hypothermia.

In certain embodiments, the treatment of a JWS phenotype is effective at suppressing the electrocardiogramd/or arrhythmic manifestations of JWS. In another embodiment, treatment of a JWS phenotype reduces instances of ventricular fibrillation and/or sudden cardiac death and/or the amplitude of J waves compared to an untreated control

In another aspect, the at least one I_{TO} inhibitor and/or I_{NA} augmentor is selected from Table 1. In another aspect, the at least one I_{TO} inhibitor and/or I_{NA} augmentor is AR-787. In certain embodiments, the at least one I_{TO} inhibitor and/or I_{NA} augmentor is administered via enteral, parenteral, topical, or pulmonary administration.

In still another aspect, the treatment of a JWS phenotype further comprises administration of at least one of an I_{CA} augmentor and/or at least one β-adrenergic agonist. In certain embodiments, the at least one ICA augmentor is isoproterenol, cilostazol and/or milrinone. In certain embodiments, the treatment of a JWS phenotype further comprises administration of at least one of quinidine, bepridil, and denopamine.

In another aspect, the treatment of a JWS phenotype further comprises treatment of hypothermia. In certain embodiments, the treatment of hypothermia is at least one of least one of wrapping the patient in blankets, administering warm fluids by mouth, immersing the patient in a warm water bath, and direct warming of a patient's blood.

Still other aspects and advantages of these compositions and methods are described further in the following detailed description of the preferred embodiments thereof.

### BRIEF DESCRIPTION OF THE FIGURES

FIG. 1 shows the molecular structure of ARumenamide-787 (AR-787).

FIGS. 2A-2E demonstrate the effect of AR-787 on the transient outward current (Iₜₒ), inward calcium current (I_{Ca}) and action potential recorded from canine right ventricular epicardial myocytes. FIG. 2A shows representative action potential traces recorded in the absence (Control) and presence of AR-787 (10 μM) at a frequency of 0.5 Hz. FIG. 2B and FIG. 2C show representative traces of Iₜₒ and I_{Ca} recorded in Control and following exposure to AR-787 (10 μM). FIG. 2D and FIG. 2E show I-V relationships for peak Iₜₒ and I_{Ca} recorded under control conditions and following exposure to AR-787. All data were obtained at 37° C. Data are reported as mean ±SEM; *p<0.05, *p<0.001 vs. Control (One Way Repeated Measures ANOVA).

FIGS. 3A-3J demonstrate effects of AR-787 on I_{Kr} recorded from HEK cells stably expressing hERG. FIG. 3A: Control. FIG. 3B: Currents recorded at a test potential of +10 mV before (control, red) and 7 min after the addition of AR-787 (black). FIG. 3C: Currents recorded 7 min after the addition of 10 μM AR-787. FIGS. 3D-3F: Currents recorded at test potentials ranging from −60 to −30 mV before (control) and 7 and 10 min after the addition of AR-787. FIG. 3G: I-V relationship of tail current amplitudes recorded at −50 mV (arrow labeled P); FIG. 3H: I-V relationship of developing steady current measured at the end of the initial step; FIG. 3I: Inactivation rate determined by curve fitting a single exponential to the tail current; FIG. 3J: Activation curves determined as tail current normalized by the peak value for each test. Red traces in FIG. 3A and FIG. 3C are recorded at a test potential of +10 mV. Red traces in FIGS. 3D-3F are currents recorded at a test potential of −40 mV. Vertical calibration mark is 0.7 nA for FIGS. 3A-3C and 0.2 nA for FIGS. 3D-3F while the horizontal line is 2 sec for all. Symbols and vertical bars in FIGS. 3G-3J represent mean ±1 SEM (n=8).

FIG. 4A-4E depicts a representative trace showing AR-787-induced deceleration of fast inactivation kinetics (AR-787-gray and Control-black). FIGS. 4A and 4B show normalized inhibition in Nav1.5 after a holding potential of −120 mV and the mid-voltage potential, respectively. FIGS. 4A and 4B show that AR-787 significantly (p<0.0001) increases normalized inhibition in Nav1.5 at −120 mV and the mid-voltage potential compared to vehicle control, respectively. FIGS. 4C and 4D show normalized τ inactivation in Nav1.5 after a holding potential of −120 mV and the mid-voltage potential, respectively. FIG. 4C shows that AR-787 significantly (p<0.0001) increases normalized τ inactivation from a holding potential of −120 mV compared to vehicle control and mid-voltage potential (FIG. 4D). FIG. 4E shows the percent of persistent current at 5 ms into the depolarizing test pulse following a holding potential of −120 mV. AR-787 significantly (p<0.0001) increases persistent current in Nav1.5 compared to vehicle control.

FIG. 5A-5C. Effect of 1, 10 and 50 μM of AR-787 to suppress the electrocardiogramanifestations in models of JWS (ERS and BrS). From top to bottom, all columns (Col) in each FIG. shows simultaneously recorded transmembrane action potentials from the M and Epi regions together with a pseudo-ECG from 3 different wedge preparations (FIG. 5A [LV wedge 1], FIG. 5B [LV wedge 2] and FIG. 5C [RV wedge]). BCL=800 ms. FIG. 5A: Control recordings (Col 1). Recordings after 20 min of perfusion with NS5806 (10 μM) (Col 2). Recordings after 25 min of addition of AR-787 (1 μM) to the perfusate (Col 3). FIG. 5B: Control recordings (Col 1). Recordings after 20 min of perfusion with the Iₜₒ agonist NS5806 (10 μM) (Col 2). Recording after 20 min of addition of AR-787 (10 μM) to the perfusate (Col 3). FIG. 5C: Control recordings (Col 1). Recording after 20 and 40 min of perfusion with NS5806 (10 μM) (Col 2 and Col 3, respectively). Recorded 15 min after addition of AR-787 (50 μM) to the perfusate (Col 4).

FIG. 6A-6C. Effect of AR-787 (10 μM) following NS5806 (5-10 μM) on AP and ECG parameters recorded from 3 LV wedge preparations. FIG. 6A: Epi Ph1 magnitude as a % of R wave amplitude. FIG. 6B: J wave amplitude as a % of R wave amplitude. FIG. 6C: QT interval. BCL=800 ms (LV wedges; n=3). NS5806 (5-10 μM) significantly increased the Epi Ph1 magnitude and the simultaneously recorded electrocardiogra wave. AR-787 (10 μM) reversed this effect restoring these parameters to control values. The QT interval was unaffected by either intervention. Data are reported as mean ±SEM; *p<0.005 vs. Control and AR-787 (One Way Repeated Measures ANOVA).

FIG. 7A-7D. Effect of AR-787 to suppress the electrocardiogramand arrhythmic manifestations in a model of JWS (ERS). From top to bottom, each panel of FIG. 7A-7D shows simultaneously recorded transmembrane action potentials from the M and Epi regions together with a pseudo-ECG from a LV wedge preparation. BCL=800 ms. FIG. 7A: Control recordings. FIG. 7B: Recordings obtained after 40 min of perfusion with a combination of NS5806 (10 μM) and verapamil (2.5 μM). FIG. 7C: Recordings obtained 22 min after the recordings shown in FIG. 7B. A closely coupled premature extrasystole is seen to trigger a short run of pVT. FIG. 7D: Recordings obtained 30 min after the addition of AR-787 (10 μM) to the coronary perfusate.

FIG. 8. Illustration of an epicardial AP, showing the basis for measurement of Phase 1 (Ph1) magnitude (measured as a % of the amplitude of Phase 0), J wave amplitude (quantified from its onset to the peak), and QT interval (measured from the onset of the QRS to the end of T wave determined by the tangent method [intersection of the maximum downslope of the T wave to the isoelectric line]). *p<0.005 vs. Control and AR-787 (One Way Repeated Measures ANOVA).

FIG. 9A-9C. Effect of AR-787 to suppress hypothermia-induced arrhythmogenesis.

### DETAILED DESCRIPTION OF THE INVENTION

The KCND3-encoded KV4.3 transient outward potassium current (Iₜₒ) plays a pivotal role in the pathophysiology of J wave syndromes,^{3,4} owing to its ability to accentuate the action potential (AP) notch and to suppress the epicardial AP dome.

An ion-channel specific and cardio-selective Iₜₒ blocker has long been sought for the management of JWS.² As described herein, a novel compound ARumenamide-787 (AR-787), via its action to inhibit Iₜₒ, can effectively suppress the electrocardiograma arrhythmic manifestations of JWS.

The effects of AR-787 on I_{Na} and I_{Kr} were studied in HEK-293 cells stably expressing the α- and β1-subunits of the cardiac (Nav1.5) sodium channel and hERG channel, respectively. In addition, the effect was studied on Iₜₒ and I_{Ca} from dissociated canine ventricular myocytes along with action potentials from coronary perfused RV and LV wedge preparations. The Iₜₒ agonist, NS5806 (5-10 μM), and the calcium channel blocker verapamil (2.5 μM) were used to mimic the genetic defects and underlying conditions associated with JWS (prominent J waves/ST segment elevation and pVT/VF).

AR-787 (1-50 μM) exerted pleiotropic effects on the cardiac ion channels. The predominant effect was inhibition of the transient outward current (Iₜₒ), with lesser effects to inhibit I_{Kr}, slow the inactivation of sodium channel current (I_{Na}) and augment calcium channel current (I_{Ca}). AR-787 (1-50 μM) diminished the electrocardiogramave and at the higher concentrations prevented or suppressed all arrhythmic activity in canine RV and LV experimental models of BrS and ERS. These findings identify AR-787 as beneficial in the pharmacologic treatment of JWS.

The present subject matter may be understood more readily by reference to the following detailed description which forms part of this disclosure. It is to be understood that this invention is not limited to the specific products, methods, conditions or parameters described and/or shown herein, and that the terminology used herein for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. In addition to definitions included in this sub-section, further definitions of terms are interspersed throughout the text.

In this invention, “a” or “an” means “at least one” or “one or more,” etc., unless clearly indicated otherwise by context. The term “or” means “and/or” unless stated otherwise. In the case of a multiple-dependent claim, however, use of the term “or” refers back to more than one preceding claim in the alternative only.

A “sample” refers to a sample from a subject that may be tested. The sample may comprise cells, and it may comprise body fluids, such as blood, serum, plasma, cerebral spinal fluid, urine, saliva, tears, pleural fluid, and the like.

As used herein the term “wild type” is a term of the art understood by skilled persons and means the typical form of an organism, strain, gene or characteristic as it occurs in nature as distinguished from mutant or variant forms. As used herein the term “variant” should be taken to mean the exhibition of qualities that have a pattern that deviates from the wild type or a comprises non naturally occurring components.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The term does not denote a particular age or sex. Thus, adult and newborn subjects, as well as fetuses, whether male or female, are intended to be covered.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action. The terms “agent” and “test compound” denote a chemical compound, a mixture of chemical compounds, a biological macromolecule, or an extract made from biological materials such as bacteria, plants, fungi, or animal (particularly mammalian) cells or tissues.

It is also contemplated that the term “compound” or “compounds” refers to the compounds discussed herein and includes precursors and derivatives of the compounds, including acyl-protected derivatives, and pharmaceutically acceptable salts of the compounds, precursors, and derivatives. The invention also includes prodrugs of the compounds, pharmaceutical compositions including the compounds and a pharmaceutically acceptable carrier, and pharmaceutical compositions including prodrugs of the compounds and a pharmaceutically acceptable carrier.

As used herein, the term “prodrug” refers to a protected form of the compound, which release the compound after administration to a subject. For example, a compound may carry a protective group which is split off by hydrolysis in body fluids, e.g., in the bloodstream, thus releasing the active compound or is oxidized or reduced in body fluids to release the compound. Accordingly, a “prodrug” is meant to indicate a compound that may be converted under physiological conditions or by solvolysis to a biologically active compound of the present disclosure. Thus, the term “prodrug” refers to a metabolic precursor of a compound of the present disclosure that is pharmaceutically acceptable. A prodrug may be inactive when administered to a subject in need thereof, but may be converted in vivo to an active compound of the present disclosure. Prodrugs are typically rapidly transformed in vivo to yield the parent compound of the present disclosure, for example, by hydrolysis in blood. The prodrug compound often offers advantages of solubility, tissue compatibility or delayed release in a subject.

A compound of the present disclosure may be administered in the form of a pharmaceutically acceptable salt. In such cases, pharmaceutical compositions in accordance with this present disclosure may comprise a salt of such a compound, preferably a physiologically acceptable salt, which are known in the art. In some embodiments, the term “pharmaceutically acceptable salt” as used herein means an active ingredient as described herein used in the form of a salt thereof, particularly where the salt form confers on the active ingredient improved pharmacokinetic properties as compared to the free form of the active ingredient or other previously disclosed salt form.

A “pharmaceutically acceptable salt” may include both acid and base addition salts. A “pharmaceutically acceptable acid addition salt” refers to those salts which retain the biological effectiveness and properties of the free bases, which are not biologically or otherwise undesirable, and which may be formed with inorganic acids such as hydrochloric acid, hydrobromic add, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

A “pharmaceutically acceptable base addition salt” refers to those salts which may retain the biological effectiveness and properties of the free acids, which may not be biologically or otherwise undesirable. These salts may be prepared from addition of an inorganic base or an organic base to the free acid. Salts derived from inorganic bases may include, but are not limited to, the sodium, potassium, lithium, ammonium, calcium, magnesium, iron, zinc, copper, manganese, aluminum salts and the like. Preferred inorganic salts may be the ammonium, sodium, potassium, calcium, and magnesium salts. Salts derived from organic bases may include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylgiucamine, theobromine, purines, piperazine, piperidine, N-ethylpiperidine, polyamine resins and the like. Particularly preferred organic bases may be isopropylamine, diethylamine, ethanolamine, trimethylamine, dicydohexylamine, choline and caffeine.

Thus, the term “pharmaceutically acceptable salt” encompasses all acceptable salts including but not limited to acetate, lactobionate, benzenesulfonate, laurate, benzoate, malate, bicarbonate, maleate, bisulfate, mandelate, bitartarate, mesylate, borate, methylbromide, bromide, methylnitrite, calcium edetate, methylsulfate, camsylate, mucate, carbonate, napsyiate, chloride, nitrate, davulanate, N-methylglucamine, citrate, ammonium salt, dihydrochloride, oleate, edetate, oxalate, edisylate, pamoate (embonate), estoiate, palmitate, esylate, pantothenate, fumarate, phosphate/diphosphate, gluceptate, polygalacturonate, gluconate, salicylate, glutame, stearate, glycollylarsanilate, sulfate, hexylresorcinate, subacetate, hydradamine, succinate, hydrobromide, tannate, hydrochloride, tartrate, hydroxynaphthoate, teoclate, iodide, tosylate, isothionate, triethiodide, lactate, panoate, valerate, and the like.

Pharmaceutically acceptable salts of a compound of the present disclosure may be used as a dosage for modifying solubility or hydrolysis characteristics, or may be used in sustained release or prodrug formulations. Also, pharmaceutically acceptable salts of a compound of this present disclosure may include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzyiethylene˜diamine, chloroprocaine, diethanolamine, procaine, N-benzylphenethyl-amine, diethylamine, piperazine, tris(hydroxymethyl)aminomethane, and tetramethylammonium hydroxide.

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly(lactic-co-glycolic acid), peptide, and polypeptide groups. Such alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, poly (lactic-co-glycolic acid), peptide, and polypeptide groups can be further substituted. Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

“Alkyl,” as used herein, refers to the radical of saturated aliphatic groups, including straight-chain alkyl, alkenyl, or alkynyl groups, branched-chain alkyl, cycloalkyl (alicyclic), alkyl substituted cycloalkyl groups, and cycloalkyl substituted alkyl. In preferred forms, a straight chain or branched chain alkyl has 30 or fewer carbon atoms in its backbone (e.g., C₁-C₃₀ for straight chains, C₃-C₃₀ for branched chains), preferably 20 or fewer, more preferably 15 or fewer, most preferably 10 or fewer. Alkyl includes methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like.

Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls,” the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred forms, a substituent designated herein as alkyl is a lower alkyl.

“Alkyl” includes one or more substitutions at one or more carbon atoms of the hydrocarbon radical as well as heteroalkyls. Suitable substituents include, but are not limited to, halogens, such as fluorine, chlorine, bromine, or iodine; hydroxyl; —NRR′, wherein R and R′ are independently hydrogen, alkyl, or aryl, and wherein the nitrogen atom is optionally quaternized; —SR, wherein R is hydrogen, alkyl, or aryl; —CN; —NO₂; —COOH; carboxylate; —COR, —COOR, or —CON(R)₂, wherein R is hydrogen, alkyl, or aryl; azide, aralkyl, alkoxyl, imino, phosphonate, phosphinate, silyl, ether, sulfonyl, sulfonamido, heterocyclyl, aromatic or heteroaromatic moieties, haloalkyl (such as —CF₃, —CH₂—CF₃, —CCl₃); —CN; —NCOCOCH₂CH₂, —NCOCOCHCH; —NCS; and combinations thereof.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), haloalkyls, —CN and the like. Cycloalkyls can be substituted in the same manner.

“Heteroalkyl,” as used herein, refers to straight or branched chain, or cyclic carbon-containing radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized.

The terms “alkoxyl” or “alkoxy,” “aroxy” or “aryloxy,” generally describe compounds represented by the formula —OR^{v}, wherein R^{v} includes, but is not limited to, substituted or unsubstituted alkyl, alkenyl, alkynyl, cycloalkyl, heterocyclyl, cycloalkenyl, heterocycloalkenyl, aryl, heteroaryl, arylalkyl, heteroalkyls, alkylaryl, alkylheteroaryl.

The terms “alkoxyl” or “alkoxy” as used herein refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. The term alkoxy also includes cycloalkyl, heterocyclyl, cycloalkenyl, heterocycloalkenyl, and arylalkyl having an oxygen radical attached to at least one of the carbon atoms, as valency permits. A “lower alkoxy” group is an alkoxy group containing from one to six carbon atoms.

The term “substituted alkoxy” refers to an alkoxy group having one or more substituents replacing one or more hydrogen atoms on one or more carbons of the alkoxy backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The term “alkenyl” as used herein is a hydrocarbon group of from 2 to 24 carbon atoms and structural formula containing at least one carbon-carbon double bond.

The term “alkynyl group” as used herein is a hydrocarbon group of 2 to 24 carbon atoms and a structural formula containing at least one carbon-carbon triple bond.

The term “aryl” as used herein is any C₅-C₂₆ carbon-based aromatic group, fused aromatic, fused heterocyclic, or biaromatic ring systems. Broadly defined, “aryl,” as used herein, includes 5-, 6-, 7-, 8-, 9-, 10-, 14-, 18-, and 24-membered single-ring aromatic groups, including, but not limited to, benzene, naphthalene, anthracene, phenanthrene, chrysene, pyrene, corannulene, coronene, etc. “Aryl” further encompasses polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “substituted aryl” refers to an aryl group, wherein one or more hydrogen atoms on one or more aromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfonamido, sulfonyl, heterocyclyl, alkylaryl, haloalkyl (such as CF₃, —CH₂—CF₃, —CCl₃), —CN, aryl, heteroaryl, and combinations thereof.

“Heterocycle,” “heterocyclic,” and “heterocyclyl” are used interchangeably, and refer to a cyclic radical attached via a ring carbon or nitrogen atom of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, C1-C10 alkyl, phenyl or benzyl, and optionally containing 1-3 double bonds and optionally substituted with one or more substituents. Heterocyclyl are distinguished from heteroaryl by definition.

Examples of heterocycles include, but are not limited to piperazinyl, piperidinyl, piperidonyl, 4-piperidonyl, dihydrofuro[2,3-b]tetrahydrofuran, morpholinyl, piperazinyl, piperidinyl, piperidonyl, 4-piperidonyl, piperonyl, pyranyl, 2H-pyrrolyl, 4H-quinolizinyl, quinuclidinyl, tetrahydrofuranyl, 6H-1,2,5-thiadiazinyl. Heterocyclic groups can optionally be substituted with one or more substituents as defined above for alkyl and aryl.

The term “heteroaryl” refers to C₅-C₂₆-membered aromatic, fused aromatic, biaromatic ring systems, or combinations thereof, in which one or more carbon atoms on one or more aromatic ring structures have been substituted with a heteroatom. Suitable heteroatoms include, but are not limited to, oxygen, sulfur, and nitrogen. Broadly defined, “heteroaryl,” as used herein, includes 5-, 6-, 7-, 8-, 9-, 10-, 14-, 18-, and 24-membered single-ring aromatic groups that may include from one to four heteroatoms, for example, pyrrole, furan, thiophene, imidazole, oxazole, thiazole, triazole, tetrazole, pyrazole, pyridine, pyrazine, pyridazine and pyrimidine, and the like. The heteroaryl group may also be referred to as “aryl heterocycles” or “heteroaromatics”. “Heteroaryl” further encompasses polycyclic ring systems having two or more rings in which two or more carbons are common to two adjoining rings (i.e., “fused rings”) wherein at least one of the rings is heteroaromatic, e.g., the other cyclic ring or rings can be cycloalkyls, cycloalkenyls, cycloalkynyls, aryls, heterocycles, or combinations thereof. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined below for “substituted heteroaryl”.

The term “substituted heteroaryl” refers to a heteroaryl group in which one or more hydrogen atoms on one or more heteroaromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfonamido, sulfonyl, heterocyclyl, alkylaryl, haloalkyl (such as CF₃, —CH₂—CF₃, —CCl₃), —CN, aryl, heteroaryl, and combinations thereof.

The term “substituted alkenyl” refers to alkenyl moieties having one or more substituents replacing one or more hydrogen atoms on one or more carbons of the hydrocarbon backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The term “substituted alkynyl” refers to alkynyl moieties having one or more substituents replacing one or more hydrogen atoms on one or more carbons of the hydrocarbon backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “aralkyl” as used herein is an aryl group having an alkyl, alkynyl, or alkenyl group as defined above attached to the aromatic group. An example of an aralkyl group is a benzyl group.

The term “hydroxyalkyl group” as used herein is an alkyl, alkenyl, alkynyl, aryl, aralkyl, cycloalkyl, halogenated alkyl, or heterocycloalkyl group described above that has at least one hydrogen atom substituted with a hydroxyl group.

The term “alkoxyalkyl group” is defined as an alkyl, alkenyl, alkynyl, aryl, aralkyl, cycloalkyl, halogenated alkyl, or heterocycloalkyl group described above that has at least one hydrogen atom substituted with an alkoxy group described above.

“Carbonyl,” as used herein, is art-recognized and includes such moieties as can be represented by the general formula:

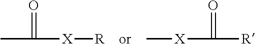

wherein X is a bond, or represents an oxygen or a sulfur, and R represents a hydrogen, a substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R″, or a pharmaceutical acceptable salt, R′ represents a hydrogen, substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl or —(CH₂)ₘ—R″; R″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. Where X is oxygen and R is defined as above, the moiety is also referred to as a carboxyl group. When X is oxygen and R is hydrogen, the formula represents a ‘carboxylic acid’. Where X is oxygen and R′is hydrogen, the formula represents a ‘formate’. Where X is oxygen and R or R′is not hydrogen, the formula represents an “ester”. In general, where the oxygen atom of the above formula is replaced by a sulfur atom, the formula represents a ‘thiocarbonyl’ group. Where X is sulfur and R or R′is not hydrogen, the formula represents a ‘thioester.’ Where X is sulfur and R is hydrogen, the formula represents a ‘thiocarboxylic acid.’ Where X is sulfur and R′is hydrogen, the formula represents a ‘thioformate.’ Where X is a bond and R is not hydrogen, the above formula represents a ‘ketone.’ Where X is a bond and R is hydrogen, the above formula represents an ‘aldehyde.’

The term “substituted carbonyl” refers to a carbonyl, as defined above, wherein one or more hydrogen atoms in R, R′ or a group to which the moiety

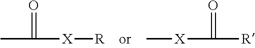

is attached, are independently substituted. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The term “carboxyl” is as defined above for the formula

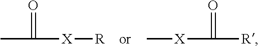

and is defined more specifically by the formula —R^{iv}COOH, wherein R^{iv} is an alkyl, alkenyl, alkynyl, cycloalkyl, heterocyclyl, alkylaryl, arylalkyl, aryl, or heteroaryl. In preferred forms, a straight chain or branched chain alkyl, alkenyl, and alkynyl have 30 or fewer carbon atoms in its backbone (e.g., C₁-C₃₀ for straight chain alkyl, C₃-C₃₀ for branched chain alkyl, C₂-C₃₀ for straight chain alkenyl and alkynyl, C₃-C₃₀ for branched chain alkenyl and alkynyl), preferably 20 or fewer, more preferably 15 or fewer, most preferably 10 or fewer. Likewise, preferred cycloalkyls, heterocyclyls, aryls and heteroaryls have from 3-10 carbon atoms in their ring structure, and more preferably have 5, 6 or 7 carbons in the ring structure.

The term “substituted carboxyl” refers to a carboxyl, as defined above, wherein one or more hydrogen atoms in R^{iv} are substituted. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof

The term “phenoxy” is art recognized, and refers to a compound of the formula —ORv wherein Rv is (i.e., —O—C₆H₅). One of skill in the art recognizes that a phenoxy is a species of the aroxy genus.

The term “substituted phenoxy” refers to a phenoxy group, as defined above, having one or more substituents replacing one or more hydrogen atoms on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The terms “aroxy” and “aryloxy,” as used interchangeably herein, are represented by —O-aryl or —O-heteroaryl, wherein aryl and heteroaryl are as defined herein.

The terms “substituted aroxy” and “substituted aryloxy,” as used interchangeably herein, represent —O-aryl or —O-heteroaryl, having one or more substituents replacing one or more hydrogen atoms on one or more ring atoms of the aryl and heteroaryl, as defined herein. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The term “alkylthio” refers to an alkyl group, as defined above, having a sulfur radical attached thereto. The “alkylthio” moiety is represented by —S-alkyl. Representative alkylthio groups include methylthio, ethylthio, and the like. The term “alkylthio” also encompasses cycloalkyl groups having a sulfur radical attached thereto.

The term “substituted alkylthio” refers to an alkylthio group having one or more substituents replacing one or more hydrogen atoms on one or more carbon atoms of the alkylthio backbone. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The term “phenylthio” is art recognized, and refers to —S—C₆H₅, i.e., a phenyl group attached to a sulfur atom.

The term “substituted phenylthio” refers to a phenylthio group, as defined above, having one or more substituents replacing a hydrogen on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

“Arylthio” refers to —S-aryl or —S-heteroaryl groups, wherein aryl and heteroaryl as defined herein.

The term “substituted arylthio” represents —S-aryl or —S-heteroaryl, having one or more substituents replacing a hydrogen atom on one or more ring atoms of the aryl and heteroaryl rings as defined herein. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The terms “amide” or “amido” are used interchangeably, refer to both “unsubstituted amido” and “substituted amido” and are represented by the general formula:

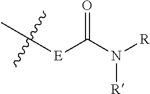

wherein, E is absent, or E is substituted or unsubstituted alkyl, substituted or unsubstituted alkenyl, substituted or unsubstituted alkynyl, substituted or unsubstituted aralkyl, substituted or unsubstituted cycloalkyl, substituted or unsubstituted aryl, substituted or unsubstituted heteroaryl, substituted or unsubstituted heterocyclyl, wherein independently of E, R and R′ each independently represent a hydrogen, substituted or unsubstituted alkyl, substituted or unsubstituted alkenyl, substituted or unsubstituted alkynyl, substituted or unsubstituted carbonyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R′″, or R and R′ taken together with the N atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred forms, only one of R and R′ can be a carbonyl, e.g., R and R′ together with the nitrogen do not form an imide. In preferred forms, R and R′ each independently represent a hydrogen atom, substituted or unsubstituted alkyl, a substituted or unsubstituted alkenyl, or —(CH₂)ₘ—R′″. When E is oxygen, a carbamate is formed. The carbamate cannot be attached to another chemical species, such as to form an oxygen-oxygen bond, or other unstable bonds, as understood by one of ordinary skill in the art.

The term “sulfonyl” is represented by the formula

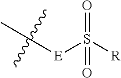

wherein E is absent, or E is alkyl, alkenyl, alkynyl, aralkyl, alkylaryl, cycloalkyl, aryl, heteroaryl, heterocyclyl, wherein independently of E, R represents a hydrogen, substituted or unsubstituted alkyl, substituted or unsubstituted alkenyl, substituted or unsubstituted alkynyl, substituted or unsubstituted amine, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R″′, or E and R taken together with the S atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred forms, only one of E and R can be substituted or unsubstituted amine, to form a “sulfonamide” or “sulfonamido.” The substituted or unsubstituted amine is as defined above.

The term “substituted sulfonyl” represents a sulfonyl in which E, R, or both, are independently substituted. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The term “sulfonic acid” refers to a sulfonyl, as defined above, wherein R is hydroxyl, and E is absent, or E is substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl.

The term “sulfate” refers to a sulfonyl, as defined above, wherein E is absent, oxygen, alkoxy, aroxy, substituted alkoxy or substituted aroxy, as defined above, and R is independently hydroxyl, alkoxy, aroxy, substituted alkoxy or substituted aroxy, as defined above. When E is oxygen, the sulfate cannot be attached to another chemical species, such as to form an oxygen-oxygen bond, or other unstable bonds, as understood by one of ordinary skill in the art.

The term “sulfonate” refers to a sulfonyl, as defined above, wherein E is oxygen, alkoxy, aroxy, substituted alkoxy or substituted aroxy, as defined above, and R is independently hydrogen, substituted or unsubstituted alkyl, substituted or unsubstituted alkenyl, substituted or unsubstituted alkynyl, substituted or unsubstituted amine, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R′″, R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. When E is oxygen, sulfonate cannot be attached to another chemical species, such as to form an oxygen-oxygen bond, or other unstable bonds, as understood by one of ordinary skill in the art.

The term “sulfamoyl” refers to a sulfonamide or sulfonamide represented by the formula

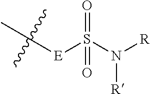

wherein E is absent, or E is substituted or unsubstituted alkyl, substituted or unsubstituted alkenyl, substituted or unsubstituted alkynyl, substituted or unsubstituted aralkyl, substituted or unsubstituted alkylaryl, substituted or unsubstituted cycloalkyl, substituted or unsubstituted aryl, substituted or unsubstituted heteroaryl, substituted or unsubstituted heterocyclyl, wherein independently of E, R and R′ each independently represent a hydrogen, substituted or unsubstituted alkyl, substituted or unsubstituted alkenyl, substituted or unsubstituted alkynyl, substituted or unsubstituted carbonyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R″′, or R and R′ taken together with the N atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred forms, only one of R and R′ can be a carbonyl, e.g., R and R′ together with the nitrogen do not form an imide.

The term “phosphonyl” is represented by the formula

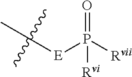

wherein E is absent, or E is substituted or unsubstituted alkyl, substituted or unsubstituted alkenyl, substituted or unsubstituted alkynyl, substituted or unsubstituted aralkyl, substituted or unsubstituted alkylaryl, substituted or unsubstituted cycloalkyl, substituted or unsubstituted aryl, substituted or unsubstituted heteroaryl, substituted or unsubstituted heterocyclyl, wherein, independently of E, R^{vi} and R^{vi} are independently hydrogen, substituted or unsubstituted alkyl, substituted or unsubstituted alkenyl, substituted or unsubstituted alkynyl, substituted or unsubstituted carbonyl, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R′″, or R and R′ taken together with the P atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8.

The term “substituted phosphonyl” represents a phosphonyl in which E, R^{vi} and R^{vii} are independently substituted. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The term “phosphoryl” defines a phosphonyl in which E is absent, oxygen, alkoxy, aroxy, substituted alkoxy or substituted aroxy, as defined above, and independently of E, R^{vi} and R^{vii} are independently hydroxyl, alkoxy, aroxy, substituted alkoxy or substituted aroxy, as defined above. When E is oxygen, the phosphoryl cannot be attached to another chemical species, such as to form an oxygen-oxygen bond, or other unstable bonds, as understood by one of ordinary skill in the art. When E, R^{vi} and R^{vii} are substituted, the substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The term “polyaryl” refers to a chemical moiety that includes two or more aryls, heteroaryls, and combinations thereof. The aryls, heteroaryls, and combinations thereof, are fused, or linked via a single bond, ether, ester, carbonyl, amide, sulfonyl, sulfonamide, alkyl, azo, and combinations thereof.

The term “substituted polyaryl” refers to a polyaryl in which one or more of the aryls, heteroaryls are substituted, with one or more substituents including, but not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The term “C₃-C₂₀ cyclic” refers to a substituted or unsubstituted cycloalkyl, substituted or unsubstituted cycloalkenyl, substituted or unsubstituted cycloalkynyl, substituted or unsubstituted heterocyclyl that have from three to 20 carbon atoms, as geometric constraints permit. The cyclic structures are formed from single or fused ring systems. The substituted cycloalkyls, cycloalkenyls, cycloalkynyls and heterocyclyls are substituted as defined above for the alkyls, alkenyls, alkynyls and heterocyclyls, respectively.

The term “ether” as used herein is represented by the formula AOA¹, where A and A¹ can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “urethane” as used herein is represented by the formula —OC(O)NRR′, where R and R′ can be, independently, hydrogen, an alkyl, alkenyl, alkynyl, aryl, aralkyl, cycloalkyl, halogenated alkyl, or heterocycloalkyl group described above.

The term “silyl group” as used herein is represented by the formula —SiRR′R″, where R, R′, and R″ can be, independently, hydrogen, an alkyl, alkenyl, alkynyl, aryl, aralkyl, cycloalkyl, halogenated alkyl, alkoxy, or heterocycloalkyl group described above.

The terms “hydroxyl” and “hydroxy” are used interchangeably and are represented by —OH.

The terms “thiol” and “sulfhydryl” are used interchangeably and are represented by —SH.

The term “oxo” refers to ═O bonded to a carbon atom.

The terms “cyano” and “nitrile” are used interchangeably to refer to —CN.

The term “nitro” refers to —NO₂.

The term “phosphate” refers to —O—PO₃.

The term “azide” or “azido” are used interchangeably to refer to —N₃.

The disclosed compounds and substituent groups, can, independently, possess two or more of the groups listed above. The compounds and substituents can be substituted with, independently, with the substituents described above.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound.

The term “inhibit” means to reduce or decrease in activity or expression. This can be a complete inhibition or activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “preventing” as used herein refers to administering a compound prior to the onset of clinical symptoms of a disease or conditions so as to prevent a physical manifestation of aberrations associated with the disease or condition.

The term “in need of treatment” as used herein refers to a judgment made by a caregiver (e.g., physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the disclosed compounds.

By “treatment” and “treating” is meant the medical management of a subject with the intent to cure, ameliorate, or stabilize, a pathological condition or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; and supportive treatment, that is, treatment employed to supplement another specific therapy directed toward the improvement of the associated disease, pathological condition, or disorder. It is understood that treatment, while intended to cure, ameliorate, or stabilize, a disease, pathological condition, or disorder, need not actually result in the cure, ameliorization, or stabilization. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

The phrase “J wave” refers to an electrocardiogram (ECG) manifestation characterized by a late delta wave following the QRS or as a small secondary R wave.

The phrase “ST segment” encompasses the region of the ECG that connects the QRS complex and the T wave. The ST segment generally has a duration of 0.005 to 0.150 seconds.

The phrase “J Wave Syndrome” or “JWS” refers to a spectrum of variable phenotypes characterized by the appearance of prominent electrocardiogramaves (or Osborn waves) with a risk of ventricular fibrillation (VF). JWS include Brugada syndrome (BrS) and Early repolarization syndrome (ERS). Although they may bear differences with regard to the electrocardiogram (ECG) lead location, amplitude, and underlying causes of J wave, these disease entities share a similar ionic and cellular basis, risk factors, and similar clinical outcomes. Although originally thought to be a benign entity, ERS is now recognized to have malignant component. BrS was initially referred to as idiopathic ventricular fibrillation (VF) associated with sudden cardiac death (SCD). Although symptoms of JWS are generally severe, it is not uncommon for patients to appear asymptomatic prior to VF and SCD manifestation.

The phrase “Brugada syndrome” or “BrS” refers to a distinct form of JWS that can lead to syncope, cardiac arrest and sudden cardiac death (SCD). The syndrome is a genetic form of cardiac rhythm disorder caused by an inherited ion channelopathy. Brugada syndrome has been observed in the electrocardiograms of otherwise healthy young individuals without evidence of structural heart disease.

The prognosis in Brugada syndrome is poor. Although the exact incidence of SCD due to Brugada syndrome is unknown, the magnitude of the problem has been estimated to range from 180,000 to 450,000 deaths annually in the United States alone. Further, about 2.5% of all cardiac arrest cases in which the patient showed no clinically identifiable cardiac abnormalities have been attributed to Brugada syndrome. The syndrome also accounts for 4% to 12% of all SCDs in genetically pre-disposed individuals, and a 40% mortality rate has been observed in symptomatic patients at two to three years follow up, with a 2% to 4% mortality rate in asymptomatic patients. During electrophysiologic studies (EPS), asymptomatic patients with induced ventricular tachycardia (VT) or ventricular fibrillation (VF) exhibited four times more SCD than non-inducible patients.

The phrase “Early repolarization syndrome” or “ERS” refers to a JWS with symptoms and complications substantially identical to those of Brugada syndrome. In ERS, J-point elevation manifests as either QRS slurring (at the transition from the QRS segment to the ST-segment) or notching (a positive deflection inscribed on terminal S wave), ST segment elevation with upper concavity in at least two contiguous leads.

ERS is commonly seen in athletes, cocaine users, hypertrophic obstructive cardiomyopathy and defects and/or hypertrophy of interventricular septal defects. The prevalence of ERS varies between 3% and 24% in the general population, depending on the population studied and methods used for ECG interpretation. Young individuals, especially those predisposed to vagotonia, males, African Americans, and athletes are subpopulations known to have a higher prevalence of ERS.

The term “hypothermia” refers to the clinical state of subnormal temperature when the body is unable to generate sufficient heat to effectively maintain normal functions. Many variables contribute to the development of hypothermia. Age, health, nutrition, body size, exhaustion, exposure, duration of exposure, wind, temperature, wetness, medication and intoxicants may decrease heat production, increase heat loss, or interfere with thermostability. In certain embodiments, hypothermia refers to a core temperature of less than 35° C. The healthy individual's compensatory responses to heat loss via conduction, convection, radiation, evaporation and respiration may be overwhelmed by exposure. J-Waves are commonly found in patients with profound hypothermia which predisposes the patient to VF and SCD.

Therapeutic hypothermia is a treatment used for people who suffered a cardiac arrest wherein the heart suddenly stops beating. Once the heart starts beating again, healthcare providers use cooling devices to lower body temperature to 32°-34° C. for a period of time, usually about 24 hours in order to avoid brain damage and neurological deficits. Therapeutic hypothermia is very helpful for some people, but it is associated with some risks, including the development of life-threatening arrhythmias due to the development of accentuated J waves.

The phrase “Sudden cardiac death” or “SCD” refers to natural death due to cardiac causes in a person who may or may not have previously recognized heart disease but in whom the time and mode of death are unexpected. In the context of time, “sudden” is defined for most clinical and epidemiologic purposes as 1 hour or less between a change in clinical status heralding the onset of the terminal clinical event and the cardiac arrest itself.

The phrase “polymorphic ventricular tachycardia” refers to a form of ventricular tachycardia in which there are multiple ventricular foci with the resultant QRS complex varying in amplitude, axis, and duration. The most common cause of PVT is myocardial ischaemia/infarction. It will either terminate spontaneously or deteriorate into ventricular fibrillation, causing cardiac arrest.

The phrase “ventricular fibrillation” or “VF” refers to a type of abnormal heart rhythm (arrhythmia). During ventricular fibrillation, disorganized heart signals cause the lower heart chambers (ventricles) to twitch (quiver) uselessly. As a result, the heart doesn't pump blood to the rest of the body. Ventricular fibrillation is caused by a problem in the heart's electrical properties which may be the result of a disruption of the normal blood supply to the heart muscle. VF is the most frequent cause of sudden cardiac death.

The phrase “cardiac action potential” or “AP” refers to a brief change in voltage (membrane potential) across the cell membrane of heart cells caused by the movement of charged atoms (called ions) between the inside and outside of the cell, through proteins called ion channels. The cardiac action potential differs from action potentials found in other types of electrically excitable cells, such as nerves. Action potentials also vary within the heart; this is due to the presence of different ion channels in cells in different regions of the heart. The cardiac action potential initially propagates through the atria stimulating muscle cells of the atrial myocardium to depolarize and contract in unison, after which the cardiac action potential encounters the atrioventricular node located at the juncture of the atria and ventricles near the center of the heart. The atrioventricular node slightly delays cardiac action potential propagation to ensure complete drainage of blood from the atria after which the muscle cells of the ventricular myocardium are stimulated resulting in systolic contraction and thereby complete the heartbeat cycle. The electrical activation of the heart can be measured using an ECG.

The phrase “cardiac transient outward potassium current” or “Iₜₒ” refers to one of the ion currents across the cell membrane of heart muscle cells. It is a result of the movement of positively charged potassium (K+) ions from the intracellular to the extracellular space. Iₜₒ has been shown to play a crucial role in shaping the early phase of repolarization (phase 1).

Iₜₒ is activated after the fast increase of the membrane potential following the phase 0 of the cardiac action potential caused by entry of sodium ions into the cell. Once activated, (K⁺) ions from inside the cells flow to the extracellular space. This outward flow of positively charged ions constitutes the Iₜₒ and causes the transmembrane voltage to decrease. This decrease of the transmembrane potential is known as repolarization. Iₜₒ is then quickly deactivated, stopping the repolarization and ending phase 1 of the action potential.

The phrase “inward calcium channel current” or “I_{CA}” refers to entry of calcium ions into the cells during phase 2 or plateau of the action potential. The entry of calcium ions into the cardiac cell causes release of calcium ion stores from the sarcoplasmic reticulum, which leads to contraction of the heart.

The phrase “Cardiac sodium channel current” or “I_{NA}” refers to entry of sodium ions into the cells during phase 0 of the action potential.

The phrase “delayed rectifier potassium current” or “I_{Kr}” refers to movement of potassium current out of a cell with a delay after membrane depolarization. Cardiac I_{Kr} channels conduct outward potassium currents during the plateau phase of the action potential and play pivotal roles in cardiac repolarization. Unlike the Iₜₒ currents that terminate quickly, the slower potassium channel current persist during the plateau phase, contribute to the repolarization of the cell and eventually terminate the action potential.

### Electrocardiogram and J Wave Syndromes

An electrocardiogram (ECG or EKG) contains three main components: the P wave, which represents the depolarization of the atria; the QRS complex, which represents the depolarization of the ventricles, and the T wave, which represents the repolarization of the ventricles. The ST segment connects the region between the QRS complex and the T wave and represents the isoelectric period when the ventricles are between depolarization and repolarization. The ST segment is connected to the QRS complex at the J-point. A J wave is a positive deflection at the J point.

J Wave Syndromes (JWS) are a class of variable phenotypes that are characterized by the appearance of prominent electrocardiogramaves. Although there may be differences with regard to the electrocardiogram (ECG) lead location, amplitude, and underlying causes of J wave, these disease entities share a similar ionic and cellular basis, risk factors, and similar clinical outcomes. In certain embodiments, JWS results in idiopathic ventricular fibrillation (VF) and sudden cardiac death (SCD). In certain embodiments, the patient is asymptomatic prior to VF and/or SCD. In certain embodiments, JWS is caused by early repolarization, Brugada syndrome, hypercalcemia, or hypothermia.

Clinical diagnosis of JWS can occur in a variety of ways. Commonly, JWS is diagnosed incidentally after detecting J waves in an asymptomatic patient. In certain embodiments, the asymptomatic patient is an athlete. In certain embodiments, the prevalence of J waves in an asymptomatic athlete increases as their training intensifies. In certain embodiments, the patient is diagnosed after surviving VF.

In certain embodiments, patients with risk factors associated with JWS are screened for J waves and diagnosed accordingly. In certain embodiments, JWS are identified by EKG. In certain embodiments, the risk factors associated with JWS have a genetic predisposition, family history of SCD or VF, being male, and being of Asian descent.

In certain embodiments, treatment of JWS includes a reduction in J waves, or an amelioration of JWS symptoms. In certain embodiments, treatment of JWS prevents VF and/or SCD.

### JWS and Hypothermia

In certain embodiments, patients with hypothermia have been shown to exhibit the JWS phenotype including the development of life-threatening arrhythmias due to the development of accentuated J waves. A patient has hypothermia if they have a core temperature of less than 35 degrees Celsius. In certain embodiments, the patient has mild hypothermia. Mild hypothermia occurs when the patient has a core temperature between 34-35 degrees Celsius. The patient is still alert and able to help him/herself and intense shivering begins. The patient's movements, however, become less coordinated and the coldness creates some pain and discomfort.

In certain embodiments, the patient has moderate hypothermia. Moderate hypothermia occurs when the patient's core temperature is between 31-33 degrees Celsius. Shivering slows or stops, muscles begin to stiffen and mental confusion and apathy sets in. Speech becomes slow, vague and slurred, breathing becomes slow and shallow, and drowsiness and strange behavior may occur.

In certain embodiments, the patient has severe hypothermia. Severe hypothermia occurs when the patient's core temperature drops below 31 degrees Celsius. Skin is cold, may be bluish-gray in color, eyes may be dilated. The patient is very weak, displays a marked lack of coordination, slurred speech, appears exhausted, may appear to be drunk, denies there is a problem and may resist help. There is a gradual loss of consciousness. There may be little or no apparent breathing, the patient may be very rigid, unconscious, and may appear dead.

In certain embodiments, the patient is intentionally given hypothermia to improve recovery after a traumatic event, such as a cardiac arrest. During this therapeutic hypothermia, healthcare providers use cooling devices to lower your body temperature to 32°-34° C. for a period of time, usually about 24 hours in order to avoid brain damage and neurological deficits.

In certain embodiments, treatment of the JWS phenotype occurs in a patient with hypothermia. In certain embodiments, the patient is never diagnosed with a JWS but is treated for JWS to ameliorate the JWS phenotype caused by hypothermia. In certain embodiments, the patient is treated for the JWS phenotype prior to being treated with therapeutic hypothermia.

In certain embodiments, the treatment of JWS in a patient with hypothermia further comprises the treatment of hypothermia. Simple methods for treating hypothermia have been known since very early times. Such methods include wrapping the patient in blankets, administering warm fluids by mouth, and immersing the patient in a warm water bath. Other devices allow for the direct warming of a patient's blood. These methods involve removing blood from the patient, warming the blood in external warming equipment, and delivering the blood back into the patient. Finally, special catheters are used for the direct warming of a patient's blood.

### Methods of Treatment

Provided herein are methods of treatment of J Wave Syndromes and arrhythmias associated with hypothermia. The methods include administration of an effective amount of at least one I_{TO} inhibitor to a subject in need thereof. In certain embodiments, the I_{TO} inhibitor is selected from those in Table 1. In some embodiments, the instance of J waves is reduced, as compared to a control.

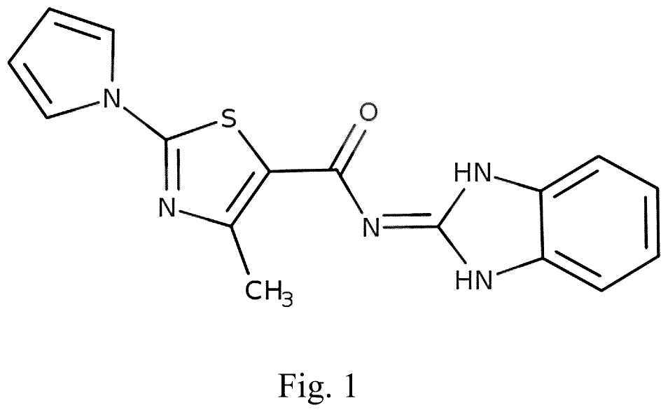

In certain embodiments, the I_{TO} inhibitor is ARumenamide-787 (AR-787), or a pharmaceutically acceptable salt, prodrug, enantiomer, solvate, or derivative thereof. The structure of AR-787 can be found in FIG. 1, and Table 1 below. The chemical name of AR-787 is N-(1H-benzimidazol-2-yl)-4-methyl-2-pyrrol-1-yl-thiazole-5-carboxamide.

TABLE 1

| | | | xlogP | | | |
|---|---|---|---|---|---|---|
| ZINC | | | (PH | MW_ | Charge_ | tPSA |
| ID | Chemical Name | Chemical Structure | 7 | PH 7 | PH 7 | (Å²) |
| ZINC 1232 3863 | N-(1H-benzimidazol- 2-yl)-4-methyl-2- pyrrol-1-yl-thiazole-5- carboxamide | 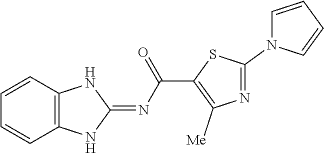 [SMILES: Cc1nc(-n2cccc2)sc1C(=O)N=c1[nH]c2ccccc2[nH]1] | 3.01 | 323.38 | 0 | 79 |
| ZINC 3853 2069 | N-(4-ethylphenyl)-2- oxo-benzimidazole-5- sulfonamide | 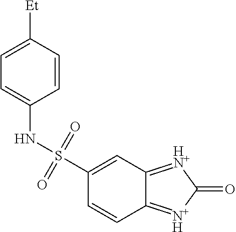 | 2.7 | 317.37 | 2 | 92 |
| ZINC 3876 7171 | N-(2-furylmethyl)-2,3- dioxo-quinoxaline 6- carboxamide | 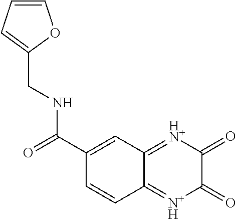 | 0.43 | 285.259 | 2 | 105 |
| ZINC 3876 7647 | N-(3-imidazol-1- ylpropyl)-2,3-dioxo- quinoxaline-6- carboxamide | 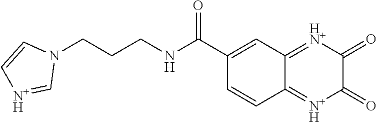 | −0.28 | 314.325 | 3 | 111 |
| ZINC 3877 6626 | N-(2-cyclohexen-1- ylethyl)-2,3-dioxo- quinoxaline-6- carboxamide | 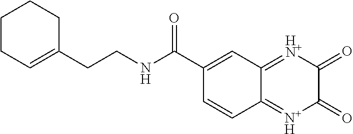 | 2.15 | 313.357 | 2 | 92 |
| ZINC 3941 1748 | 6-(4-methylpiperazin- 1- yl)sulfonylquinoxaline- 2,3-dione | 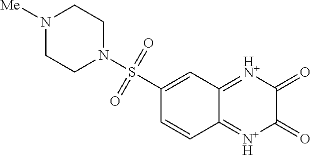 | -0.22 | 324.362 | 2 | 103 |
| ZINC 3969 9427 | N-allyl-2-oxo- benzimidazole-5- sulfonamide | 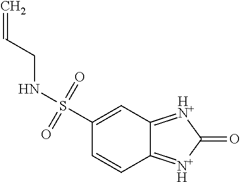 | 0.73 | 253.283 | 2 | 92 |
| ZINC 3970 9283 | N-(m-tolyl)-2,3-dioxo- quinoxaline-6- sulfonamide | 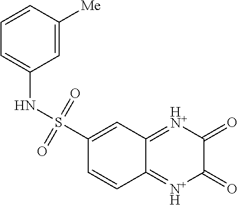 | 1.77 | 331.353 | 2 | 109 |
| ZINC 3972 9455 | N-(3-ethylphenyl)-2- oxo-benzimidazole-5- sulfonamide | 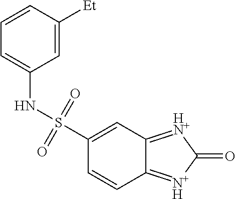 | 2.68 | 317.37 | 2 | 92 |
| ZINC 3977 6444 | N-ethyl-N-(4- fluorophenyl)-2-oxo- benzimidazole-5- sulfonamide | 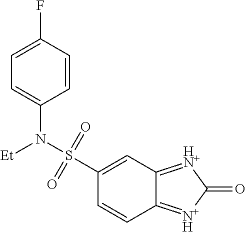 | 2.57 | 335.36 | 2 | 83 |
| ZINC 4001 4265 | N-[(2S)-2- (dimethylamino)-2-(2- furyl)ethyl]-2,3-dioxo- quinoxaline-6- carboxamide | 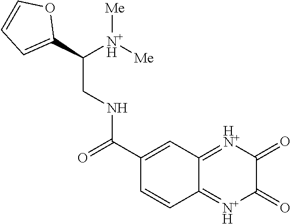 | 0.64 | 343.363 | 3 | 109 |
| ZINC 4001 4267 | N-[(2R)-2- (dimethylamino)-2-(2- furyl)ethyl]-2,3-dioxo- quinoxaline-6- carboxamide | 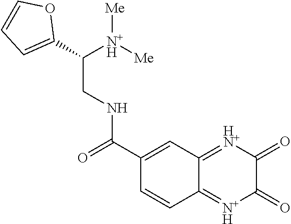 | 0.64 | 343.363 | 3 | 109 |
| ZINC 6390 8937 | N-[2-(4- methoxyphenyl)ethyl]- 2-oxo-benzimidazole- 5-sulfonamide | 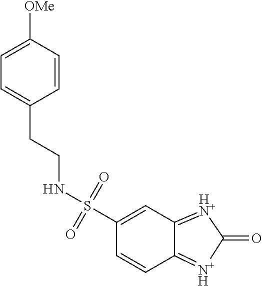 | 1.95 | 347.396 | 2 | 101 |
| ZINC 6397 0468 | N-methyl-N-[(2- methyl-3- furyl)methyl]-2-oxo- benzimidazole-5- sulfonamide | 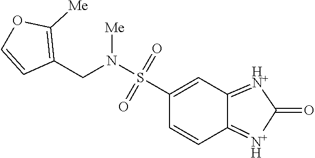 | 0.9 | 321.358 | 2 | 96 |
| ZINC 6400 2748 | 5-[2-[2- furylmethyl(methyl) amino]acetyl] benzimidazol-2-one | 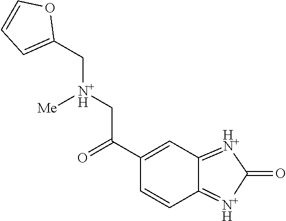 | 1.23 | 286.311 | 3 | 80 |
| ZINC 6401 2417 | N-(1-methylpyrazol-3- yl)-2,3-dioxo- quinoxaline-6- sulfonamide | 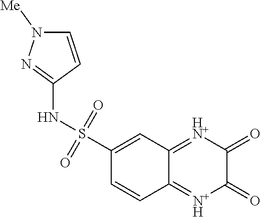 | −0.08 | 321.318 | 2 | 126 |
| ZINC 6447 0706 | N-(2-chloro-4-methyl- phenyl)-2-oxo- benzimidazole-5- sulfonamide | 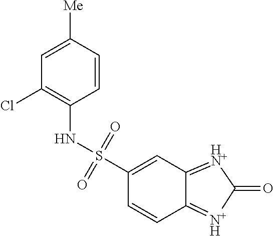 | 2.84 | 337.788 | 2 | 92 |
| ZINC 6447 0729 | N-(3-methoxyphenyl)- 2-axo-benzimidazole- 5-sulfonamide | 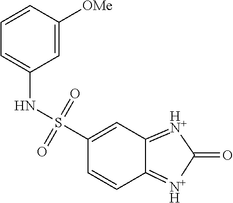 | 1.82 | 319.342 | 2 | 101 |
| ZINC 6447 0737 | N-(2-ethylphenyl)-2- oxo-benzimidazole-5- sulfonamide | 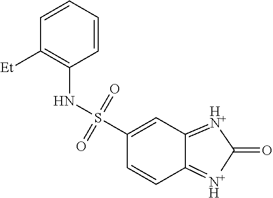 | 2.65 | 317.37 | 2 | 92 |
| ZINC 6447 0745 | N-(4-fluoro-2-methyl- phenyl)-2-oxo- benzimidazole-5- sulfonamide | 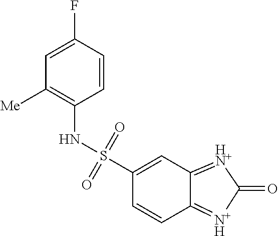 | 2.33 | 321.333 | 2 | 92 |
| ZINC 6447 0747 | N-[(2- methoxyphenyl)methyl]- 2-oxo- benzimidazole-5- sulfonamide | 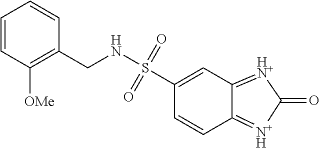 | 1.5 | 333.369 | 2 | 101 |

The compounds of Table 1 can be generally categorized into carboxamides and sulfonamides.

In certain embodiments, the method of treatment effectively suppresses the electrocardiogramanifestations of JWS. In some embodiments, the method suppresses arrhythmia associated with JWS. In some embodiments, the method suppresses ventricular fibrillation. In some embodiments, the method prevents sudden cardiac death.

In certain embodiments, treatment of J Wave Syndromes further comprises an inward shift in the balance of currents contributing to the early phases of the ventricular AP. In certain embodiments an inward shift is caused by augmenting the inward calcium channel current (I_{Ca}) by administering an effective amount of at least one I_{Ca} augmenter to a patient in need thereof. An I_{Ca} augmenter increases levels of I_{Ca}. In certain embodiments, the I_{CA} augmenter is isoproterenol. In certain embodiments, the I_{Ca} augmenter also helps inhibit Iₜₒ. In certain embodiments, these I_{Ca} augmenters include cilostazol or milrinone.

In certain embodiments, an inward shift is caused by increasing late I_{Na} during the early phases of the action potential by administering an effective amount of at least one I_{Na} augmenter to a patient in need thereof. In certain embodiments the I_{Na} augmenter is AR-787 or lithospermate B, both of which slow the inactivation of the sodium channel current, thus increasing I_{Na} during the early phases of the action potential, resulting in suppression of the J wave.

β-adrenergic agonists have been shown to suppress electrical storms and the associated accentuated J waves to help ameliorate symptoms of JWS. In certain embodiments, treatment of the J Wave Syndromes further comprises administration of an effective amount of at least one β-adrenergic agonist to a patient in need thereof. In certain embodiments, the β-adrenergic agonist is bepridil, denopamine, and/or cilostazol.

### Administration

The compounds described herein can be formulated for enteral, parenteral, topical, or pulmonary administration. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients. Typical carriers and conventional methods of preparing pharmaceutical compositions that can be used in conjunction with the preparation of formulations of the compounds are known by those skilled in the art. These compositions can take the form of solutions, suspensions, emulsion, tablets, pills, capsules, powders, sustained-release formulations and the like.

The compounds described herein can be formulated for parenteral administration. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion. Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

For intravenous administration, the compositions may be packaged in solutions of sterile isotonic aqueous buffer. Where necessary, the composition may also include a solubilizing agent. The components of the composition are supplied either separately or mixed together in unit dosage form, for example, as a dry lyophilized powder or concentrated solution in a hermetically sealed container such as an ampoule or sachet indicating the amount of active agent. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above.

The compounds described herein can be administered in an effective amount to a subject that is in need of alleviation or amelioration from one or more symptoms associated with J Wave Syndromes.

The exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation. The dosages or amounts of the compounds described herein are large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex and extent of the disease in the subject and can be determined by one of skill in the art. The dosage can be adjusted by the individual physician based on the clinical condition of the subject involved. The dose, schedule of doses and route of administration can be varied.

The compositions are administered in an effective amount and for a period of time effect to reduce one or more symptoms associated with the disease to be treated. It should be understood that the “effective amount” for a composition which comprises AR-787 or a modification thereof, may vary. In one embodiment an effective amount includes without limitation about 0.001 to about 25 mg/kg subject body weight. In one embodiment, the range of effective amount is 0.001 to 0.01 mg/kg body weight. In another embodiment, the range of effective amount is 0.001 to 0.1 mg/kg body weight. In another embodiment, the range of effective amount is 0.001 to 1 mg/kg body weight. In another embodiment, the range of effective amount is 0.001 to 10 mg/kg body weight. In another embodiment, the range of effective amount is 0.001 to 20 mg/kg body weight. In another embodiment, the range of effective amount is 0.01 to 25 mg/kg body weight. In another embodiment, the range of effective amount is 0.01 to 0.1 mg/kg body weight. In another embodiment, the range of effective amount is 0.01 to 1 mg/kg body weight. In another embodiment, the range of effective amount is 0.01 to 10 mg/kg body weight. In another embodiment, the range of effective amount is 0.01 to 20 mg/kg body weight. In another embodiment, the range of effective amount is 0.1 to 25 mg/kg body weight. In another embodiment, the range of effective amount is 0.1 to 1 mg/kg body weight. In another embodiment, the range of effective amount is 0.1 to 10 mg/kg body weight. In another embodiment, the range of effective amount is 0.1 to 20 mg/kg body weight. In another embodiment, the range of effective amount is 1 to 25 mg/kg body weight. In another embodiment, the range of effective amount is 1 to 5 mg/kg body weight. In another embodiment, the range of effective amount is 1 to 10 mg/kg body weight. In another embodiment, the range of effective amount is 10 to 20 mg/kg body weight. In another embodiment, the range of effective amount is 20 to 30 mg/kg body weight. In another embodiment, the range of effective amount is 30 to 40 mg/kg body weight. In another embodiment, the range of effective amount is 40 to 50 mg/kg body weight. In another embodiment, the range of effective amount is 1 to 50 mg/kg body weight. Still other doses falling within these ranges are expected to be useful.

In another embodiment, the range of effective amount is 0.001 mg to 10 g. In another embodiment, the range of effective amount is 0.01 mg to 1 g. In another embodiment, the range of effective amount is 0.01 mg to 100 mg. In another embodiment, the range of effective amount is 0.1 mg to 100 mg. In another embodiment, the range of effective amount is 0.1 mg to 500 mg. In another embodiment, the range of effective amount is 1 mg to 100 mg. In another embodiment, the range of effective amount is 10 mg to 500 mg. In another embodiment, the range of effective amount is 10 mg to 750 mg. In another embodiment, the range of effective amount is 0.01 mg to 100 mg. In another embodiment, the range of effective amount is 1 mg to 500 mg. In another embodiment, the effective amount is 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, 650 mg, 651 mg, 652 mg, 653 mg, 654 mg, 655 mg, 656 mg, 657 mg, 658 mg, 659 mg, 660 mg, 661 mg, 662 mg, 663 mg, 664 mg, 665 mg, 666 mg, 667 mg, 668 mg, 669 mg, 670 mg, 671 mg, 672 mg, 673 mg, 674 mg, 675 mg, 676 mg, 677 mg, 678 mg, 679 mg, 680 mg, 681 mg, 682 mg, 683 mg, 684 mg, 685 mg, 686 mg, 687 mg, 688 mg, 689 mg, 690 mg, 691 mg, 692 mg, 693 mg, 694 mg, 695 mg, 696 mg, 697 mg, 698 mg, 699 mg, 700 mg, 701 mg, 702 mg, 703 mg, 704 mg, 705 mg, 706 mg, 707 mg, 708 mg, 709 mg, 710 mg, 711 mg, 712 mg, 713 mg, 714 mg, 715 mg, 716 mg, 717 mg, 718 mg, 719 mg, 720 mg, 721 mg, 722 mg, 723 mg, 724 mg, 725 mg, 726 mg, 727 mg, 728 mg, 729 mg, 730 mg, 731 mg, 732 mg, 733 mg, 734 mg, 735 mg, 736 mg, 737 mg, 738 mg, 739 mg, 740 mg, 741 mg, 742 mg, 743 mg, 744 mg, 745 mg, 746 mg, 747 mg, 748 mg, 749 mg, 750 mg, 751 mg, 752 mg, 753 mg, 754 mg, 755 mg, 756 mg, 757 mg, 758 mg, 759 mg, 760 mg, 761 mg, 762 mg, 763 mg, 764 mg, 765 mg, 766 mg, 767 mg, 768 mg, 769 mg, 770 mg, 771 mg, 772 mg, 773 mg, 774 mg, 775 mg, 776 mg, 777 mg, 778 mg, 779 mg, 780 mg, 781 mg, 782 mg, 783 mg, 784 mg, 785 mg, 786 mg, 787 mg, 788 mg, 789 mg, 790 mg, 791 mg, 792 mg, 793 mg, 794 mg, 795 mg, 796 mg, 797 mg, 798 mg, 799 mg, 800 mg, 801 mg, 802 mg, 803 mg, 804 mg, 805 mg, 806 mg, 807 mg, 808 mg, 809 mg, 810 mg, 811 mg, 812 mg, 813 mg, 814 mg, 815 mg, 816 mg, 817 mg, 818 mg, 819 mg, 820 mg, 821 mg, 822 mg, 823 mg, 824 mg, 825 mg, 826 mg, 827 mg, 828 mg, 829 mg, 830 mg, 831 mg, 832 mg, 833 mg, 834 mg, 835 mg, 836 mg, 837 mg, 838 mg, 839 mg, 840 mg, 841 mg, 842 mg, 843 mg, 844 mg, 845 mg, 846 mg, 847 mg, 848 mg, 849 mg, 850 mg, 851 mg, 852 mg, 853 mg, 854 mg, 855 mg, 856 mg, 857 mg, 858 mg, 859 mg, 860 mg, 861 mg, 862 mg, 863 mg, 864 mg, 865 mg, 866 mg, 867 mg, 868 mg, 869 mg, 870 mg, 871 mg, 872 mg, 873 mg, 874 mg, 875 mg, 876 mg, 877 mg, 878 mg, 879 mg, 880 mg, 881 mg, 882 mg, 883 mg, 884 mg, 885 mg, 886 mg, 887 mg, 888 mg, 889 mg, 890 mg, 891 mg, 892 mg, 893 mg, 894 mg, 895 mg, 896 mg, 897 mg, 898 mg, 899 mg, 900 mg, 901 mg, 902 mg, 903 mg, 904 mg, 905 mg, 906 mg, 907 mg, 908 mg, 909 mg, 910 mg, 911 mg, 912 mg, 913 mg, 914 mg, 915 mg, 916 mg, 917 mg, 918 mg, 919 mg, 920 mg, 921 mg, 922 mg, 923 mg, 924 mg, 925 mg, 926 mg, 927 mg, 928 mg, 929 mg, 930 mg, 931 mg, 932 mg, 933 mg, 934 mg, 935 mg, 936 mg, 937 mg, 938 mg, 939 mg, 940 mg, 941 mg, 942 mg, 943 mg, 944 mg, 945 mg, 946 mg, 947 mg, 948 mg, 949 mg, 950 mg, 951 mg, 952 mg, 953 mg, 954 mg, 955 mg, 956 mg, 957 mg, 958 mg, 959 mg, 960 mg, 961 mg, 962 mg, 963 mg, 964 mg, 965 mg, 966 mg, 967 mg, 968 mg, 969 mg, 970 mg, 971 mg, 972 mg, 973 mg, 974 mg, 975 mg, 976 mg, 977 mg, 978 mg, 979 mg, 980 mg, 981 mg, 982 mg, 983 mg, 984 mg, 985 mg, 986 mg, 987 mg, 988 mg, 989 mg, 990 mg, 991 mg, 992 mg, 993 mg, 994 mg, 995 mg, 996 mg, 997 mg, 998 mg, 999 mg, or 1000 mg.

In certain embodiments, the I_{TO} inhibitor described herein is provided with one or more additional therapies for Brugada or other JWS. Treatment for BrS includes medications such as those used for treatment of fever, medications useful for treatment of abnormal heart rhythm, such as quinidine, implantable cardioverter-defibrillator (ICD), and catheter ablation.

In certain embodiments, the I_{TO} inhibitor described herein is provided with one or more additional therapies for hypothermia. Treatment for hypothermia includes forced-air warming, extracorporeal blood warming, administration of glucose, thiamine, warmed fluids, and steroids.

### EXAMPLES

The invention is now described with reference to the following examples. These examples are provided for the purpose of illustration only and the invention should in no way be construed as being limited to these examples but rather should be construed to encompass any and all variations that become evident as a result of the teaching provided herein.

### Example 1: Materials and Methods

### Canine Hearts

Ten hearts from purpose-bred adult male mongrel dogs (20-22 kg) were acquired from Covance (Denver, PA) and another 10 hearts from purpose-bred adult male Beagle dogs (10-16 kg) were obtained from Envigo (Denver, PA). Dogs were sedated with ketamine (10 mg/kg, IM) and xylazine 2 (mg/kg, IM). Prior to euthanasia (Euthasol solution: pentobarbital sodium and phenytoin sodium; 0.22 ml/kg, IV), and heparin (human pharmaceutical grade, 1,000 U/kg, IV) were administered 3-4 min before isolation of the hearts via a left thoracotomy. Retrograde aortic perfusion of the hearts with ice-cold cardioplegic solution (modified K-H solution; 16 mM KCl) was immediately performed to rinse the coronary vasculature of blood. The hearts were then placed in a sealed container containing ice-cold cardioplegic solution and transported to our institution via private courier in an insulated package filled with icepacks.

### Ventricular Wedge Preparations

Transmural wedges were dissected from the base of the RV and LV. During the cannulation procedure, preparations were initially arterially perfused with cardioplegic solution. Next, the wedge preparations were placed in a tissue bath and perfused with a modified Krebs-Henseleit solution (K—H) bubbled with 95% O₂/5% CO₂ warmed to 37° C. The composition of K—H was (in mM): 118 NaCl, 4 KCl, 2 CaCl₂, 1.2 MgSO₄, 24 NaHCO₃, 1.2 NaH2PO₄, 2 Na Pyruvate and 10 D-Glucose. The perfusate was delivered at a constant pressure (45-50 mmHg). A transmural pseudo-ECG (ECG) was recorded using two Ag/AgCl half cells placed at ˜1 cm from the Epi (+) and Endo (−) surfaces along the same axis as the transmembrane action potential (AP) recordings. APs were simultaneously recorded from the immediate sub-Epi and M cell region (˜2-3 mm from the Endo surface) using floating glass microelectrodes. Pacing was applied to the Endo surface using bipolar silver electrodes insulated except at the tips (Basic Cycle Lengths [BCLs]: 500-2,000 ms). A detailed description of the arterially-perfused ventricular wedge preparation has been previously published.⁵ ECG and AP signals were digitized and analyzed using Spike 2 for Windows (Cambridge Electronic Design, Cambridge, UK).

### Wedge Models of the J Wave Syndromes

We used the Iₜₒ-agonist NS5806 (5-10 μM) alone or with the calcium-channel blocker verapamil (2.5 μM) to pharmacologically mimic the effects of the ion channel genetic defects underlying ERS and BrS. These provocative agents were added to the coronary perfusate in increasing concentrations until the characteristic ECG phenotypes were unmasked, including phase 2 reentry (P2R), closely coupled premature beats (CCPBs) and/or pVT/VF. AR-787 was added to the perfusate to test its ability to suppress or prevent arrhythmogenesis.

### Dissociation of Canine Ventricular Myocytes

Single myocytes were isolated from coronary-perfused canine right or left ventricular wedge preparations via enzymatic dissociation. Briefly, the wedge preparations were coronary perfused with nominally Ca⁺²-free Tyrode's solution for a period of 15 min. They were then subjected to enzymatic digestion in nominally Ca²⁺-free solution supplemented with 0.6 mg/ml collagenase (type II; Worthington) and 0.075 mg/ml protease (type XIV; Sigma) for 25-35 min. The wedge preparation was then perfused for 15 min with a taurine buffer solution containing (in mM): 108 NaCl, 10 HEPES, 11 D-glucose, 4 KCl, 1.2 MgSO₄, 1.2 NaH₂PO₄, 50 taurine and 0.025 CaCl₂. pH was adjusted to 7.4 using NaOH. After perfusion, thin slices of tissue were cut from the epicardial surface to obtain single epicardial cells as previously described.⁶

### Cell Culture

Human Embryonic Kidney (HEK) cells stably expressing hERG channels (NM_000238) were used to evaluate the effect of AR-787 on I_{Kr}. The HEK cells were cultured in vented flasks using standard methods. The culture media consisted of MEM α-modified, 10% FBS, 1% L-glutamine, 1% pen-strep and 0.4% G418 (Geneticin). Cells were passaged at 50% confluence.

HEK cells transiently transfected with wild-type SCN5A (α subunit—Nav1.5) together with SCN1B (β1 subunit) using Lipofectamine 2000 were used to assess the effect of AR-787 on the cardiac sodium channel current (I_{Na}). All cells were incubated at 37° C./5% CO₂. All cell culture reagents were purchased from Thermo Fisher Scientific (Waltham, MA).

### Whole-Cell Patch Clamp Measurement of Ito, ICa and Action Potentials (APs) in Dissociated Canine Ventricular Myocytes

For electrophysiological study, cells were released from culture with trypsin, rinsed with Ca²⁺-free external solution and maintained on ice until used (up to 4 hr). An aliquot of cells was place in a chamber on an inverted microscope (Nikon Diaphot) and perfused with external solution at a rate of 1 ml/min at room temperature. The external solution contained in (mM): 140 NaCl, 5 KCl, 2 CaCl₂, 1 MgCl₂, 10 HEPES and 10 glucose at pH 7.4 (adjusted with NaOH). Membrane currents recorded with an Axopatch 200B amplifier controlled by Clampex software were digitized at a sampling rate of 10 kHz and filtered at 2 kHz. Micropipettes were fabricated from borosilicate glass and generally had resistances of 1.8-2.4 MΩ when filled with internal solution consisting of (mM): 140 KCl, 1 MgCl₂, 5 Na₂ATP, 10 HEPES and 10 EGTA (pH 7.3 adjusted with KOH; 300 mOsm). Pipette offset and stray capacitance were compensated with the pipette in the bath solution before seal formation. After achieving cell access, whole-cell capacitance and series resistance were compensated optimally. Series resistance prediction and correction were usually adjusted to 85% or higher leaving an uncompensated series resistance of less than 1 MΩ and voltage errors estimated to be less than 5 mV. Voltage protocols and currents were recorded on a computer for offline analysis using Clampfit and GraphPad software.

Iₜₒ and APs were measured in isolated RV or LV myocytes. APs were measured in the current-clamp mode in the whole-cell configuration. APs were initiated using intracellular current injection and recorded continuously by means of Clampex software (Axon Instruments/Molecular Devices) at a sampling frequency of 0.5 Hz. Iₜₒ was measured using the Axopatch 200B-2 amplifier in V-Clamp mode.

Iₜₒ was recorded in isolated ventricular epicardial cells at 37° C. using whole-cell patch clamp techniques. Iₜₒ was elicited using a series of 350 ms voltage steps between −50 mV and +40 mV from a holding potential of −90 mV. A 15 ms prepulse to −20 mV was used to discharge the sodium current. Interpulse interval was 50 ms. The amplitude of _{Ito} was determined by subtracting peak current from steady-state current at the end of the pulse. The total charge carried by Iₜₒ was determined by calculating the area under the Iₜₒ current waveform. The extracellular solution contained (in mM): 140 NaCl, 4 KCl, 1.8 CaCl₂, 1.2 MgCl₂, 10 HEPES, and 10 glucose, pH 7.4 (adjusted with NaOH). The intracellular solution contained (in mM): 120 KCl, 1.5 MgCl₂, 10 HEPES, 0.5 EGTA, 0.01 CaCl₂, 5 MgATP, pH 7.2 (adjusted with NaOH). Following baseline measurements, recordings was repeated during exposure to AR-787.

I_{Ca} was measured in canine ventricular myocytes at 37° C. I_{Ca} was elicited by application of 300 ms steps from −50 to +40 mV. Extended protocols were avoided to prevent current rundown. Internal solution contained (in mM): 120 CsCl, 1 MgCl₂, 10 EGTA, 5 Mg-ATP, 10 HEPES, 5 CaCl₂. pH was adjusted to 7.2 with CsOH. External solution contained (in mM): 140 NaCl, 10 HEPES, 10 D-glucose, 4 KCl, 1 MgCl₂, 2 CaCl₂, 2 4-AP and 0.1 BaCl₂.

I_{Kr} was measured using a two-step protocol. Voltage steps of 4 sec duration were applied at 20 sec intervals from a holding potential of −80 mV to −60 to +40 mV in 10 mV steps, followed by a 4 sec step to −50 mV to record the tail currents. Following control recordings, a step protocol was applied, and perfusion changed electronically (ALA Scientific) to an external solution containing 10 μM AR-787. The step protocol consisted of a voltage step from a holding potential of −80 mV to a test potential of +10 mV for 4 sec then to −50 mV for 4 second. It was repeated at 20 sec intervals. The step protocol was maintained until the current response achieved a stable level (5-7 min). The I-V protocol was then repeated.

For analysis, the following were determined: 1) currents averaged over the last 100 ms of the first voltage clamp step (−60 to +40 mV) and 2) peak tail current during the second voltage clamp step (at −50 mV). These were divided by cell capacitance to normalize for cell size (pA/pF). The time course of tail current inactivation was fit with a single exponential function using Clampfit. The voltage dependence of activation was determined from tail currents by dividing current value at each voltage by the maximum value for each I-V protocol. Values of current were averaged among cells and expressed as a mean ±SEM.

### Automated Patch-Clamp Experiments

The effect of AR-787 on cardiac sodium channel current was evaluated in HEK cells co-transfected with SCN5A and SCN1B. I_{Na} was measured in the whole-cell configuration using a Qube-384 (Sophion A/S, Copenhagen, Denmark) automated voltage clamp system. A flipped Na⁺ gradient was used where the extracellular solution contained (in mM): 125 choline chloride, 1 NaCl, 5 KCl, 2 CaCl₂, 1 MgCl₂, 10 HEPES/NaOH, pH 7.4 (adjusted with NaOH), and the intracellular solution contained (in mM): 115 NaF, 15 CsCl₂, 5 CsF, 3 Na₂ATP, 0.3 Na₂GTP, 2 MgCl₂, 0.1 CaCl₂, 10 EGTA, and 10 HEPES/CsOH, pH 7.2 (adjusted with NaOH). The currents were low-pass-filtered at 5 kHz and recorded at 25 kHz sampling frequency. Series resistance compensation was applied at 100%, and leak subtraction was enabled. Experiments were performed at room temperature (27° C.). Appropriate filters for cell membrane resistance (typically >500 MΩ), series resistance (<10 MΩ), and Nav current magnitude (>500 pA at a test pulse from a resting holding potential of −120 mV) were routinely applied to exclude poor quality cells. Vehicle (0.5% DMSO) controls were run on each plate to enable correction for any compound independent decrease of currents over time.

We used a double voltage-pulse protocol designed specifically to assess whether the compound is a potentiator, inhibitor, or both. The protocol assesses these parameters at both rest and partially inactivated holding potentials. Currents were assessed by a test pulse of 0 mV from a holding potential at rest (−120 mV) before preconditioning channels to their appropriate mid-potential (V_{1/2}) values (Na_{V}1.5 V_{1/2}=−90 mV) for 10 s. Peak current, time constant of inactivation (τ inactivation) and persistent current were measured.

Time constants (τ) for fast inactivation onset were derived by fitting a single exponential function to the decay of current obtained from the depolarizing test pulse following a holding potential of −120 mV.

$$I = {I}_{ss} + α exp ( - ( t - {τ}_{0} ) / τ )$$

where I is current amplitude, Iₛₛ is the plateau amplitude, α is the amplitude at time 0 for time constant τ, and t is time. Persistent sodium current was measured during a 10 ms test pulse from −120 mV to 0 mV. It was calculated as a percentage of peak sodium current at 5 ms.

### Drugs

ARumenamide-787 (MolPort-005-972-787/ZINC000012323863/Vitas-M STK638098) was purchased from MolPort in powder form. It was dissolved in 100% DMSO to create a stock solution of 10 mM and diluted with external solution to the desired final concentration. DMSO content was always <0.5%. The molecular structure of AR-787 is shown in FIG. 1. Verapamil was purchased from Sigma-Aldrich (St Louis, MO) and dissolved in 100% DMSO. Electrophysiological parameters were measured after 5 mins of exposure to the compounds tested.

### Statistical Analysis

Statistical analysis was performed using one-way repeated measures analysis of variance (ANOVA) or a two-factor completely randomized design ANOVA followed by a post hoc Tukey test. Our statistical model was a full factorial in which all the factors were allowed to interact together. Data are shown as mean ±SEM. Statistical significance was considered at p<0.05.

### Iₜₒ Inhibitors

AR-787, and other compounds of the same class, are examined for their use as Iₜₒ inhibitors.

Such compounds include, without limitation, those recited in Table 1 above and those described in International Application No. PCT/IB2020/050853, which is incorporated herein by reference in its entirety. These compounds can be generally categorized into carboxamides and sulfonamides.

### Example 2: Effect of Iₜₒ Inhibitors on JWS

We examined the effect of AR-787 in: 1) Na_{V}1.5 stably expressed in HEK293 cells, 2) KV11.1 (hERG-I_{Kr}) stably expressed in HEK293 cells, 3) KV4.3 (Iₜₒ) and CaV1.2 (I_{Ca}) in canine ventricular epicardial myocytes, 4) coronary-perfused canine right (RV) and left (LV) ventricular wedge models of JWS in which the transient outward current agonist NS5806 alone or in combination with the calcium channel blocker verapamil were used to elicit the arrhythmic phenotypes by mimicking the genetic defects underlying BrS and ERS, and 5) coronary-perfused canine right ventricular (RV) wedge models of JWS in which the current agonist NS5806 in combination with decreasing temperature were used to elicit the arrhythmic phenotypes of hypothermia.

### Results

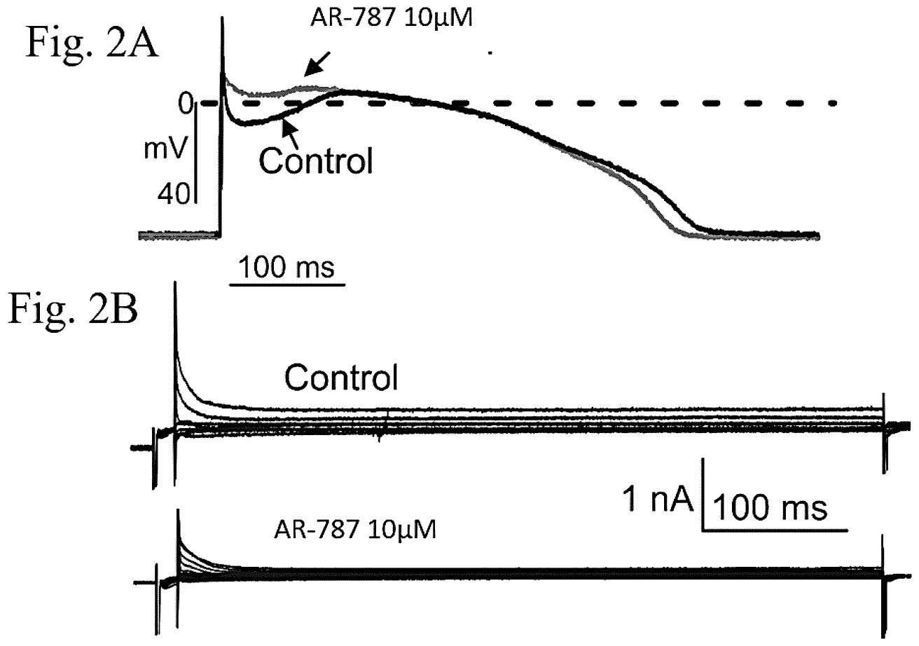

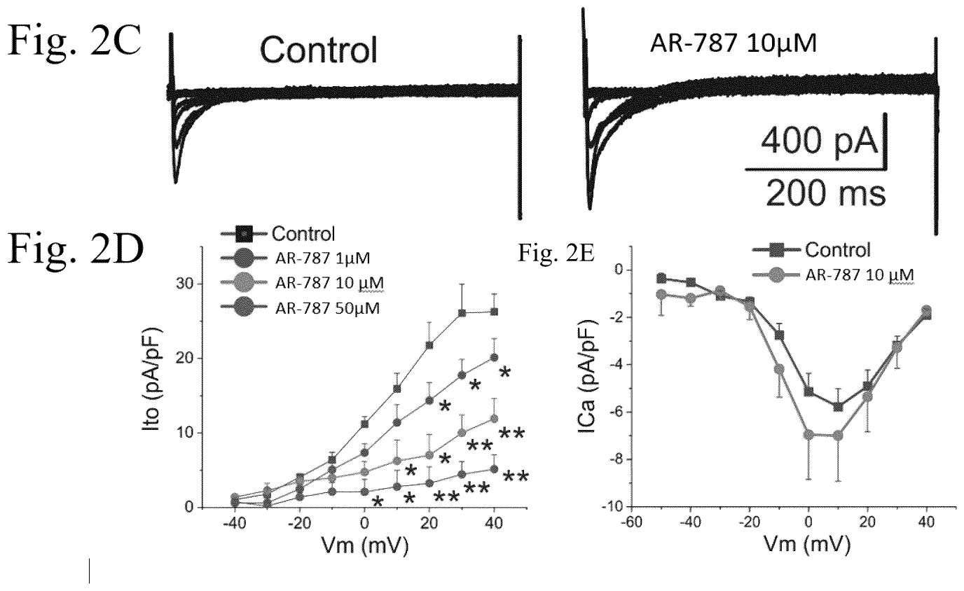

FIG. 2 illustrates the effect of AR-787 on Iₜₒ, I_{Ca} and action potential morphology recorded from enzymatically dissociated canine RV epicardial myocytes. AR-787 (10 μM) significantly suppressed the AP notch and produced a concentration-dependent inhibition of Iₜₒ. At +20 mV, and at concentrations of 1, 10 and 50 μM, AR-787 reduced Iₜₒ density by 34, 68 and 85.2%, respectively (FIG. 2B and 2D). Action potential duration (APD) displayed a tendency to abbreviate but this effect was not statistically significant. The IC₅₀ for Iₜₒ inhibition is estimated to be approximately 8 μM. AR-787 (10 μM) increased I_{Ca} peak density from −5.13+0.77 pA/pF to −6.95+1.88 pA/pF at 0 mV (FIG. 2C and FIG. 2E); however, this shift was not statistically significant.

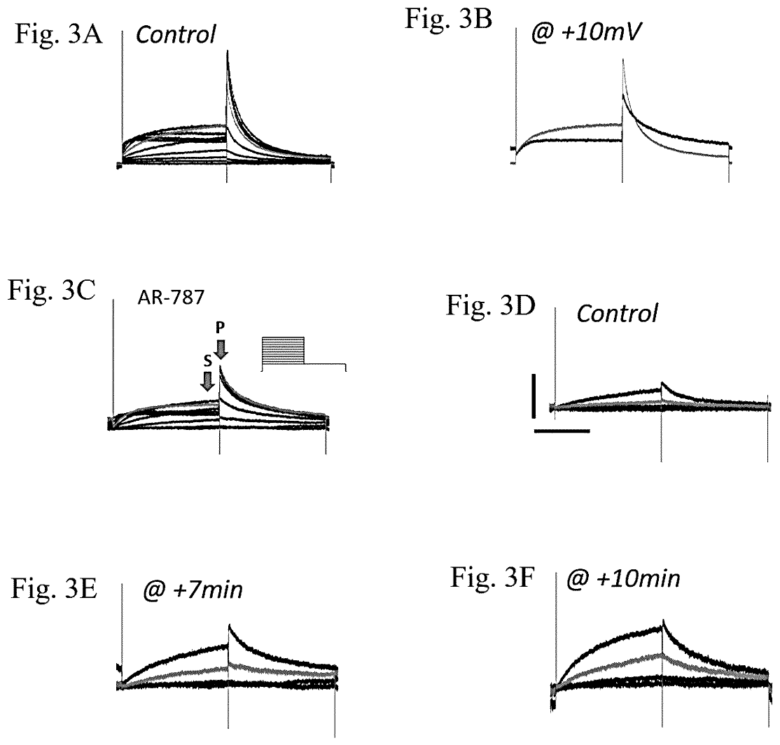

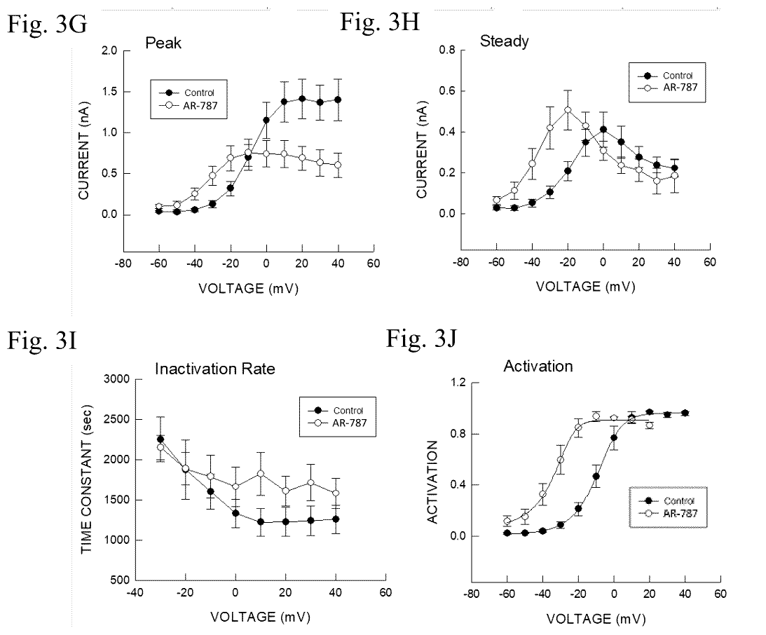

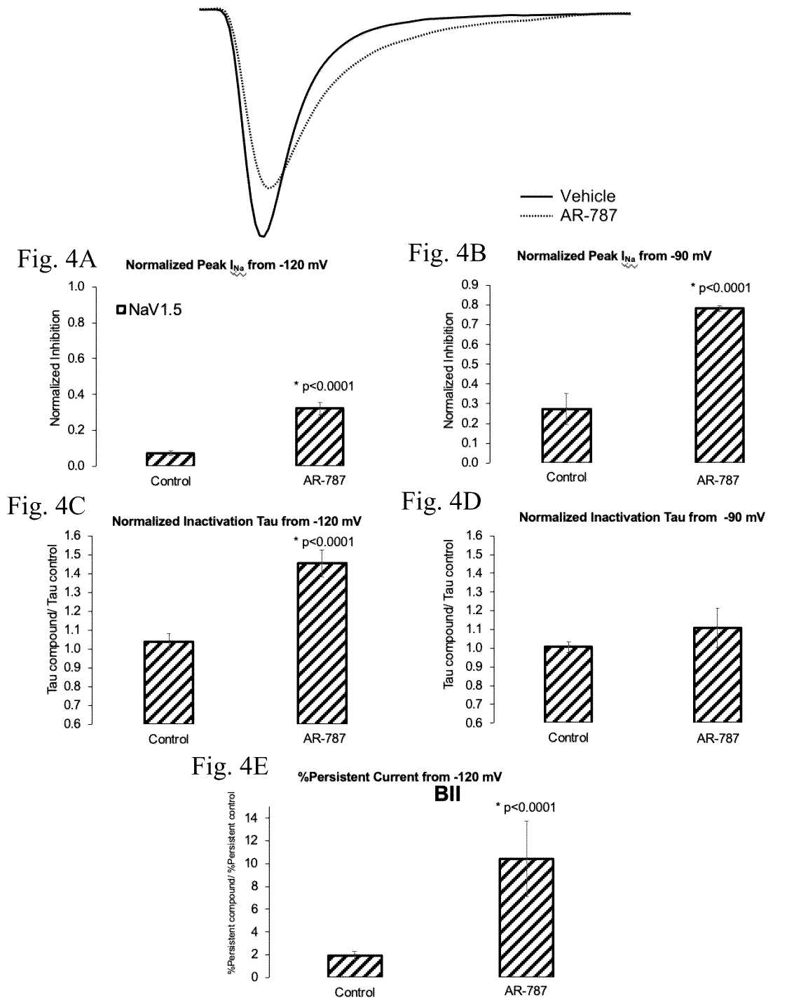

FIG. 3 shows the effect of AR-787 on I_{Kr} measured in HEK cells stably expressing hERG. AR-787 (50 μM) produced a 50% reduction in peak tail current and a −20 mV shift in the voltage dependence of both developing and tail currents (FIG. 4G and 4H). The voltage-dependence of activation shifted by −25 mV and the rates of inactivation of tail currents were significantly slowed at positive voltages (FIG. 4I). Overall, the amplitude of the developing I_{Kr} was enhanced between −60 to −20 mV and reduced above 0 mV. These effects result in an increase in I_{Kr} at voltages associated with action potential repolarization.

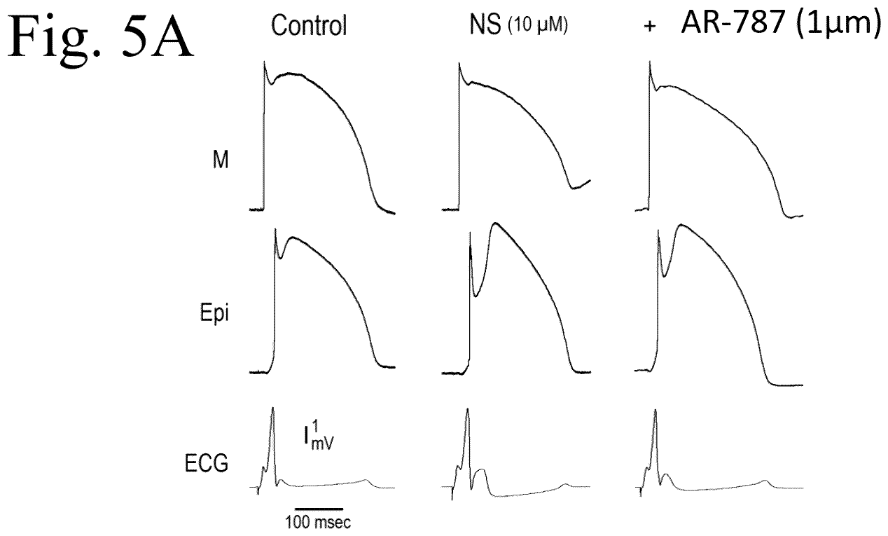

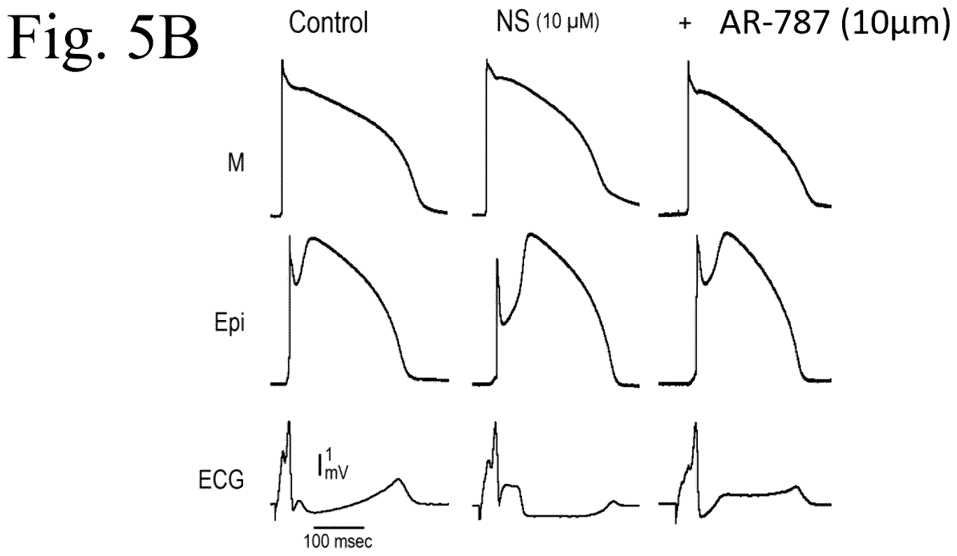

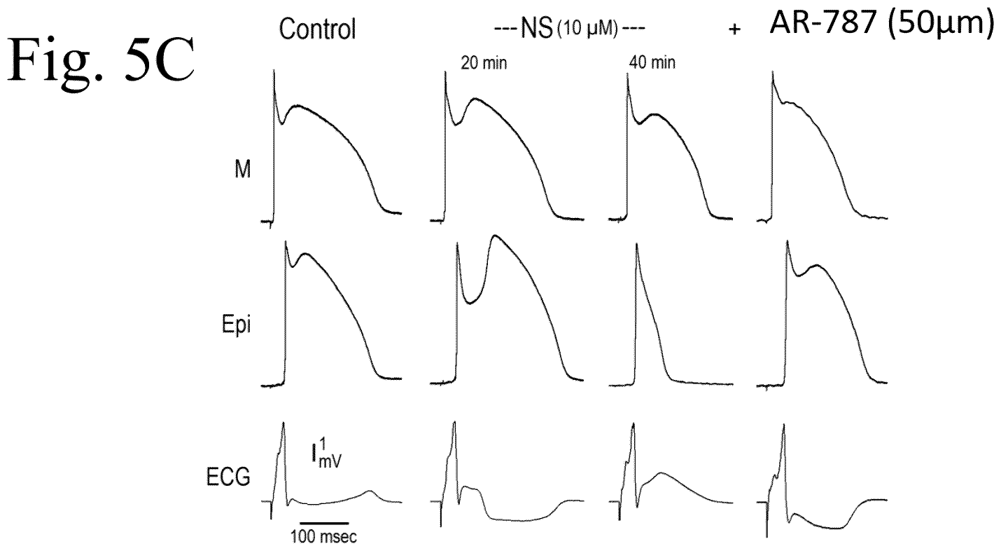

The effect of AR-787 on I_{Na} was measured in HEK cells transfected with SCN5A+SCN1B (FIG. 4). Normalized inhibition was measured by a 0 mV test pulse from a holding potential of −120 mV as shown in FIG. 5A. AR-787 (50 μM) reduced I_{Na} by ˜32% (FIG. 4A and Table 2).

TABLE 2

| Parameters | Control (VEH)_Nav1.5 | AR-787_Nav1.5 |
|---|---|---|
| Normalized Inhibition | 0.07 ± 0.01 | 0.32 ± 0.03 |
| (−120 mV) | | |
| Normalized Inhibition | 0.27 ± 0.08 | 0.78 ± 0.02 |
| (−65 mV and −90 mV) | | |
| Normalized τ_{inactivation} | 1.04 ± 0.04 | 1.45 ± 0.07 |
| (−120 mV) | | |
| Normalized τ_{inactivation} | 1.01 ± 0.03 | 1.11 ± 0.10 |
| (−65 mV and −90 mV) | | |
| Normalized AUC | 0.96 ± 0.03 | 1.03 ± 0.06 |
| Normalized Persistent | 1.93 ± 0.39 | 10.44 ± 3.30 |
| Current (5 ms) | | |

This effect was further exacerbated when the cells were depolarized from a holding potential of −90 mV. Under these conditions, AR-787 inhibited peak I_{Na} by 80% (FIG. 4B and Table 2), indicating that AR-787-induced inhibition of I_{Na} is strongly voltage dependent.

AR-787 (50 μM) was observed to decelerate fast inactivation kinetics by ˜1.5-fold (FIG. 4C and Table 2) when depolarized from a holding potential of −120 mV. When depolarized from a holding potential of −90 mV, this effect was abolished (FIG. 4D and Table 2). With this prolonged depolarization pulse, Na_{V} channels do not have sufficient time to recover from fast and slow inactivation, which indicates that AR-787 elicits its mechanism of action by binding to the channel in the resting state and not in the depolarized (inactive) state.

The amplitude of persistent sodium current (late I_{Na}; as a percent of peak current) (FIG. 4E and Table 2) was measured at 5 ms during a 10 ms depolarization to 0 mV from a holding potential of −120 mV. AR-787 increased late I_{Na} by ˜10% as predicted from the decelerated inactivation kinetics induced by the drug. The persistent current was fully attenuated by the end of the 10 ms depolarization pulse.

In another experimental series, we used canine ventricular RV and LV wedge preparations to generate experimental models of JWS by pharmacologically mimicking the effects of the associated genetic defects leading to an increase of Ito and/or to reduced levels of I_{Ca}.

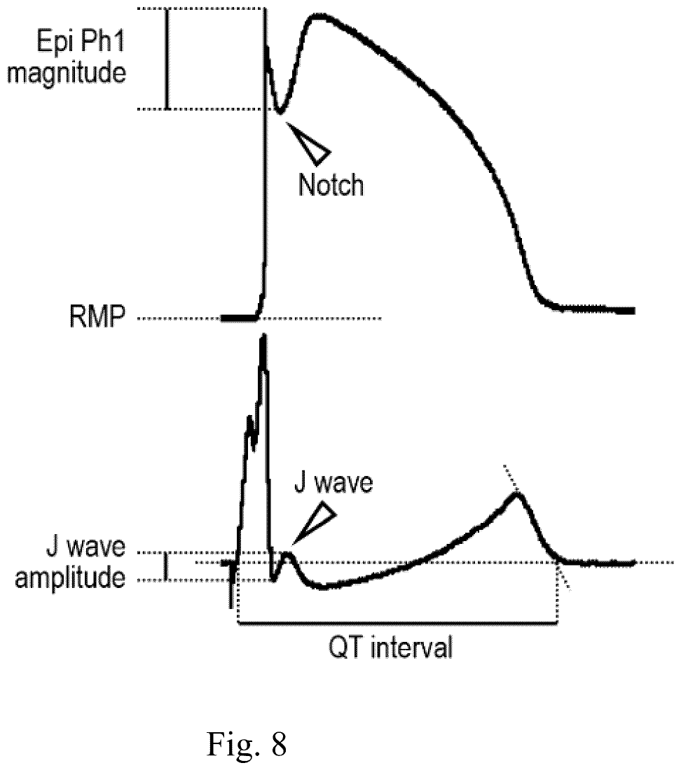

Table 3 and FIG. 8 show the effects of the Iₜₒ agonist NS5806 (NS, 5-10 μM) and of AR-787 (10 μM) on action potential and ECG parameters.

TABLE 3
The effect of NS5806 (NS, 5-10 μM) and AR-787 (10 μM)
on action potential and ECG parameters in models of JWS.

| CL 800 msec | Control | NS (5-10 μM) | AR (10 μM) |
|---|---|---|---|
| M (APD50) | 196.4 ± 5.9 | 179.5 ± 5.9 | 179.0 ± 11.4 |
| Epi (APD50) | 174.7 ± 8.1 | 181.8 ± 10.4 | 173.9 ± 7.8 |
| M (APD90) | 252.1 ± 8.4 | 228.4 ± 7.7 | 235.0 ± 11.1 |
| Epi (APD90) | 211.6 ± 8.0 | 215.6 ± 5.3 | 209.0 ± 8.1 |
| Ph1 mag (% Ph0) | 18.8 ± 7.9 | 40.2 ± 6.5* | 25.4 ± 4.9 |
| QT interval | 252.5 ± 8.3 | 261.2 ± 4.5 | 254.3 ± 10.0 |
| T wave amplitude | 22.9 ± 5.8 | 17.8 ± 9.6 | 19.0 ± 1.8 |
| J wave amplitude | 10.9 ± 4.6 | 31.3 ± 4.6* | 8.7 ± 5.7 |

*Data (M ± SE) derived from 3 LV wedge preparations.
BCL = 800 msec

Table 3 and FIG. 8 display data recorded from LV and RV wedge preparations. The introduction of 5-10 μM NS5806 to the coronary perfusate increased the magnitude of phase 1, which accentuated the AP notch and electrocardiogramave, and thus led to the development of the J wave syndrome phenotype. AR-787 (10 μM) reversed these effects induced by NS5806.

FIG. 5 shows the effect of 1, 10 and 50 μM AR-787 to suppress the electrocardiogramanifestations of JWS in models of ERS and BrS generated by exposing LV (FIG. 5A and 5B) and RV (FIG. 5C) wedge preparations to 10 μM of NS5806. From top to bottom, all columns (Col) in each panel show simultaneously recorded transmembrane action potentials from the M and Epi regions together with a pseudo-ECG. Shown are recordings obtained under control conditions, 20-40 min following exposure to 10 μM NS5806 and following the further addition of 1-50 μM AR-787 to the coronary perfusate.

The addition of NS5806 to the coronary perfusate dramatically augmented the Epi AP notch resulting in the accentuation of the J wave in the ECG, but without the development of arrhythmias (Col 2 of FIG. 5A, 5B and 5C). FIG. 5C shows loss of the Epi action potential dome after 40 min of exposure to the Iₜₒ agonist NS5806 (10 μM). AR-787 caused a concentration-dependent diminution in the manifestation of the AP notch and associated ECG J wave. It is noteworthy that several episodes of Phase 2 reentry manifest as closely coupled extrasystoles developed following 40 min of exposure to NS5806. AR-787 (50 μM but not 10 μM) was effective in fully restoring the AP dome and in totally suppressing this arrhythmic activity.

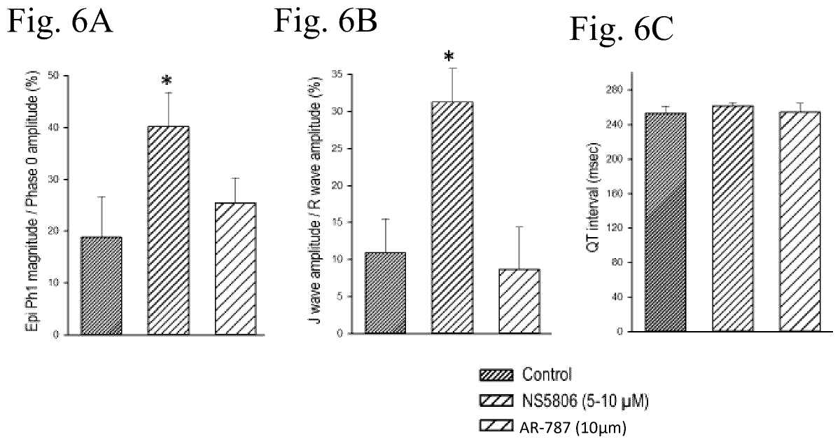

FIG. 6 summarizes the effect of AR-787 (1, 10 and 50 μM) following NS5806 (5-10 μM) on AP and ECG parameters recorded from five wedge preparations (4 LV and 1 RV). Addition of AR-787 to the coronary perfusate (1, 10 or 50 μM) readily reversed the effect of NS5806 to augment phase 1 magnitude of the epicardial AP (Epi Ph1 magnitude) as well as the dramatic increase in J wave amplitude. QT interval was unaffected by either drug.

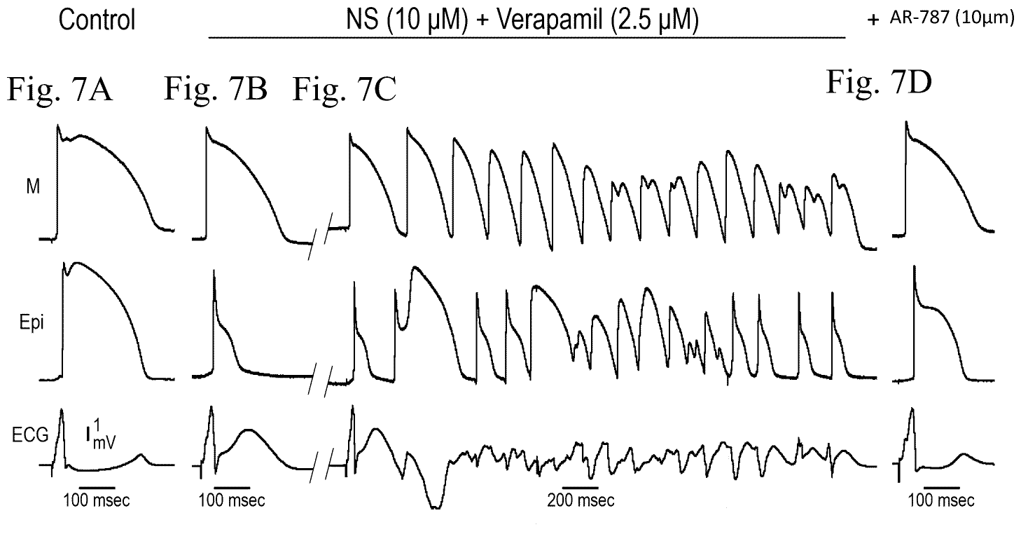

FIG. 7 shows the ability of AR-787 to suppress the ECG and arrhythmic manifestations in a model of JWS (ERS). Exposure of the LV wedge preparation to NS5806 resulted in the development of a closely coupled extrasystole following the loss of the epicardial action potential dome (FIG. 7B), most likely due to phase 2 reentry, which subsequently triggered polymorphic VT (FIG. 7C). AR-787 (10 μM) reduced Phase 1 magnitude, restored the AP dome and totally suppressed all arrhythmic activity (FIG. 7D). Primarily due to its inhibition of Iₜₒ, AR-787 was effective in suppressing the electrocardiogramd arrhythmic manifestations of JWS (FIGS. 5-7).

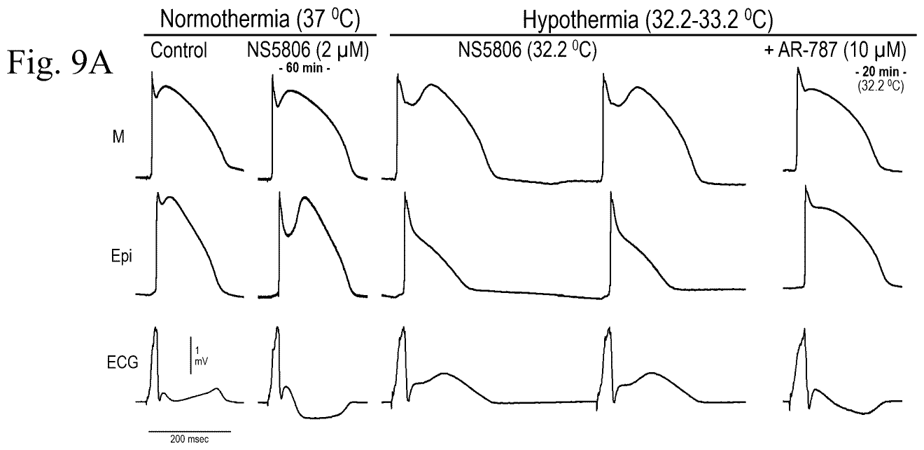

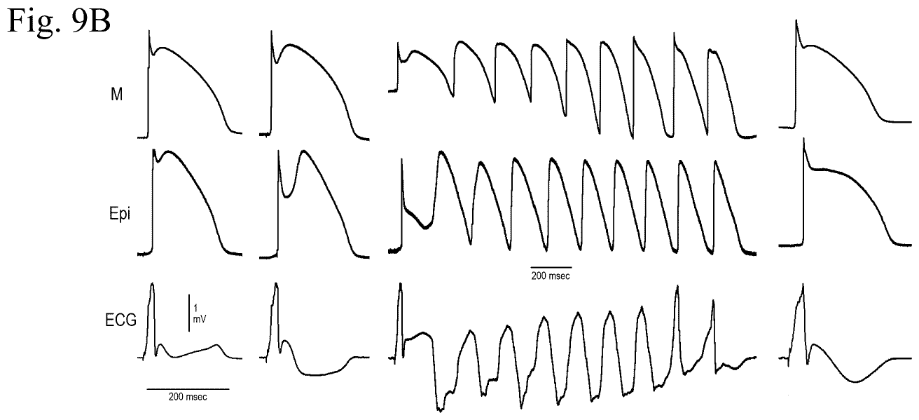

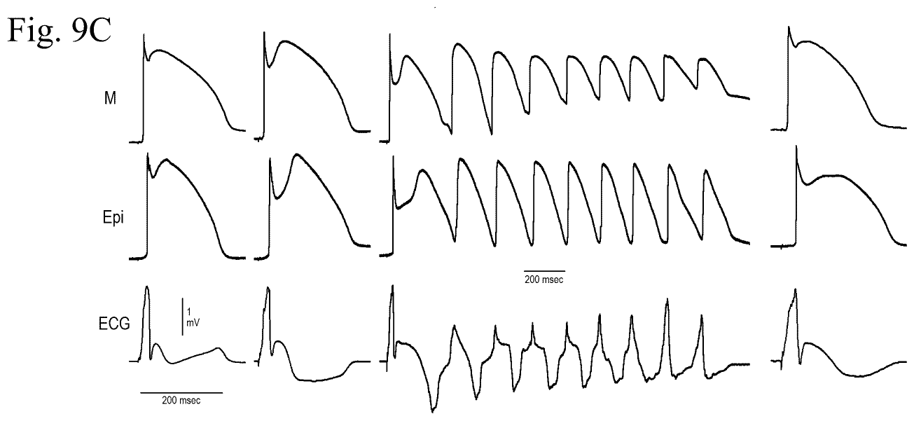

FIG. 9A-9C show the ability of AR-787 to suppress the ECG and arrhythmic manifestations in a model of hypothermia. Recordings obtained from a RV wedge preparation were obtained at a BCL of 500 (FIG. 9A), 800 (FIG. 9B) and 2000 (FIG. 9C) msec. From top to bottom, each column displays subendocardial (M) and epicardial (Epi) action potential recordings, together with a pseudo-ECG. The first shows Control recordings whereas the second column show traces recorded 60 minutes after addition of the Ito agonist NS5806 (2 μM) to the perfusate (37° C.; Normothermia). The third grouping was recorded 10 minutes after lowering the temperature of the perfusate to 32.7±0.5° C. (32.2-33.2° C.). Hypothermia led to accentuation of the electrocardiogramave, the development of phase 2 reentry as well as several episodes of polymorphic ventricular tachycardia (pVT) at BCLs 800 and 2000 msec (please note the different time scale in these 2 tracings). The fourth column was recorded 20 min after addition of AR-787 (10 μM). AR-787 diminished the action potential (AP) notch and J wave thus suppressing the repolarization abnormalities responsible for the hypothermia-induced phase 2 reentry and PVT.

### Discussion

The present Example tests the hypothesis that Iₜₒ inhibitors, such as ARumenamide-787 (AR-787), via their action to inhibit Iₜₒ can effectively suppress the electrocardiogramd arrhythmic manifestations of JWS. Our findings identify AR-787 as a new agent for pharmacologic therapy of JWS due to its unique effect to block Iₜₒ and thus prevent repolarization abnormalities underlying arrhythmogenesis in patients with JWS.

We demonstrate the effect of AR-787 at concentrations as low as 10 μM to suppress the electrocardiogramd arrhythmic manifestations of both BrS and ERS. Our findings support the hypothesis that an inward shift in the balance of currents contributing to the early phases of the ventricular AP exerts ameliorative effects on the electrocardiogramd arrhythmic manifestations of JWS. Augmenting this inward depolarizing reserve reverses the effects of the outward shift in the balance of currents in the early phase of the epicardial action potential resulting from the genetic defects underlying in JWS.⁸⁻¹⁶ Experimental approaches to therapy of both BrS and ERS have demonstrated the benefits of inhibiting outward currents such as Iₜₒ with agents such as quinidine, augmenting inward calcium channel current (I_{Ca}) using drugs such as isoproterenol, as well as combined inhibition of Iₜₒ and augmentation of I_{Ca} using agents such as cilostazol and milrinone. It is noteworthy that AR-787 produces an inward shift in the balance of current not only by suppressing Iₜₒ, but also by increasing late I_{Na} during the early phases of the action potential secondary to slowing of the inactivation of the cardiac sodium channels. This latter effect of AR-787 is similar to that previously described for lithospermate B, which has been shown to suppress arrhythmogenesis in experimental models of the J wave syndromes.^{17,18}

In the clinic, ERS and BrS have been shown to share similarities in their response to pharmacological therapy. Electrical storms (and the associated accentuated J waves) can be suppressed with β-adrenergic agonists.¹⁹⁻²² Chronic oral administration of quinidine,^{23,24} bepridil,²¹ denopamine,^{19,25} and cilostazol,^{19,21,25-29} are reported to prevent VT/VF in both syndromes.^{26,30,31} Its ameliorative effects notwithstanding, quinidine by virtue of its action to block I_{Kr} prolongs the QT interval and predisposes to development of life-threatening Torsade de Pointes (TdP) arrhythmias. High plasma levels of quinidine are required to avoid this side effect, but these high doses cause approximately 50% of patients to develop serious GI side effects. Cilostazol does not always work, and the other pharmacological approaches mentioned have limitations as well. Thus, there is a critical need for additional safe and effective agents. Our findings indicate that AR-787 produces beneficial results when used for the treatment of JWS.

### REFERENCES

1. Antzelevitch C, Yan G X. J wave syndromes. Heart Rhythm 4/2010 2010;7:549-558.
2. Antzelevitch C, Yan G X, Ackerman M J, et al. J-Wave syndromes expert consensus conference report: Emerging concepts and gaps in knowledge. Heart Rhythm Jul. 13, 2016.
3. Yan G X, Antzelevitch C. Cellular basis for the Brugada syndrome and other mechanisms of arrhythmogenesis associated with ST-segment elevation. Circulation 1999;100:1660-1666.
4. Koncz I, Gurabi Z, Patocskai B, Panama B K, Szel T, Hu D, Barajas-Martinez H, Antzelevitch C. Mechanisms underlying the development of the electrocardiogramd arrhythmic manifestations of early repolarization syndrome. J Mol Cell Cardiol Mar. 1, 2014 2014;68C:20-28.
5. Di Diego J M, Sicouri S, Myles R C, Burton F L, Smith G L, Antzelevitch C. Optical and electrical recordings from isolated coronary-perfused ventricular wedge preparations. J Mol Cell Cardiol 1/2013 2013;54:53-64.
6. Barajas-Martinez H H V, Chamberland C, Blais Roy M-J, Fecteau M H, Cordeiro J M, Dumaine R. Larger dispersion of INa in female dog ventricle as a mechanism for gender-specific incidence of cardiac arrhythmias. Cardiovascular research 2009;81:82-89.
7. Di Diego J M, Sun Z Q, Antzelevitch C. Ito and action potential notch are smaller in left vs. right canine ventricular epicardium. AmJPhysiol 1996 1996;271:H548-H561.
8. Gussak I, Antzelevitch C, Bjerregaard P, Towbin J A, Chaitman B R. The Brugada syndrome: clinical, electrophysiologic and genetic aspects. J Am Coll Cardiol 1/1999 1999;33:5-15.
9. Antzelevitch C, Yan G X. Cellular and ionic mechanisms responsible for the Brugada syndrome. JElectrocardiol 2000 2000;33 Suppl:33-39.
10. Shimizu W, Aiba T, Antzelevitch C. Specific therapy based on the genotype and cellular mechanism in inherited cardiac arrhythmias. Long QT syndrome and Brugada syndrome. CurrPharmDes 4/2005 2005;11:1561-1572.
11. Antzelevitch C, Fish J M. Therapy for the Brugada syndrome. Handb Exp Pharmacol 1/2006 2006305-330.
12. Szel T, Koncz I, Antzelevitch C. Cellular mechanisms underlying the effects of milrinone and cilostazol to suppress arrhythmogenesis associated with Brugada syndrome. Heart rhythm: the official journal of the Heart Rhythm Society 2013;10:1720-1727.
13. Patocskai B, Antzelevitch C. Novel Therapeutic Strategies for the Management of Ventricular Arrhythmias Associated with the Brugada Syndrome. Expert Opin Orphan Drugs 2015;3:633-651.
14. Antzelevitch C, Yan G X. J-wave syndromes: Brugada and early repolarization syndromes. Heart Rhythm August 2015;12:1852-1866.
15. Argenziano M, Antzelevitch C. Recent advances in the treatment of Brugada syndrome. Expert Rev Cardiovasc Ther June 2018;16:387-404.
16. Di Diego J M, Patocskai B, Barajas-Martinez H, Borbáth V, Ackerman M J, Burashnikov A, Clatot J, Li G R, Robinson V M, Hu D, Antzelevitch C. Acacetin suppresses the electrocardiogramd arrhythmic manifestations of the J wave syndromes. PloS one 2020;15:e0242747.
17. Yoon J Y, Ahn S H, Oh H, Kim Y S, Ryu S Y, Ho W K, Lee SH. A novel Na+ channel agonist, dimethyl lithospermate B, slows Na+ current inactivation and increases action potential duration in isolated rat ventricular myocytes. BrJ Pharmacol 11/2004 2004; 143:765-773.
18. Fish J M, Welchons D R, Kim Y S, Lee S H, Ho W K, Antzelevitch C. Dimethyl lithospermate B, an extract of danshen, suppresses arrhythmogenesis associated with the Brugada syndrome. Circulation Mar. 13, 2006 2006;113:1393-1400.
19. Shimizu W, Kamakura S. Catecholamines in children with congenital long QT syndrome and Brugada syndrome. J Electrocardiol 2001 2001;34 Suppl:173-175.
20. Suzuki H, Torigoe K, Numata O, Yazaki S. Infant case with a malignant form of Brugada syndrome. Journal of cardiovascular electrophysiology Nov. 20, 2000 2000; 11:1277-1280.
21. Ohgo T, Okamura H, Noda T, Satomi K, Suyama K, Kurita T, Aihara N, Kamakura S, Ohe T, Shimizu W. Acute and chronic management in patients with Brugada syndrome associated with electrical storm of ventricular fibrillation. Heart Rhythm 6/2007 2007;4:695-700.
22. Watanabe A, Fukushima K K, Morita H, Miura D, Sumida W, Hiramatsu S, Banba K, Nishii N, Nagase S, Nakamura K, Sakuragi S, Ohe T. Low-dose isoproterenol for repetitive ventricular arrhythmia in patients with Brugada syndrome. EurHeart J 7/2006 2006;27:1579-1583.
23. Hermida J S, Denjoy I, Clerc J, Extramiana F, Jarry G, Milliez P, Guicheney P, Di Fusco S, Rey J L, Cauchemez B, Leenhardt A. Hydroquinidine therapy in Brugada syndrome. J Am Coll Cardiol May 19, 2004 2004;43:1853-1860.
24. Belhassen B, Viskin S. Pharmacologic approach to therapy of Brugada syndrome: quinidine as an alternative to ICD therapy? In: Antzelevitch C, Brugada P, Brugada J, Brugada R, eds. The Brugada Syndrome: From Bench to Bedside. Oxford: Blackwell Futura; 2004:202-211.
25. Tsuchiya T, Ashikaga K, Honda T, Arita M. Prevention of ventricular fibrillation by cilostazol, an oral phosphodiesterase inhibitor, in a patient with Brugada syndrome. JCardiovascElectrophysiol 7/2002 2002;13:698-701.
26. Iguchi K, Noda T, Kamakura S, Shimizu W. Beneficial effects of cilostazol in a patient with recurrent ventricular fibrillation associated with early repolarization syndrome. Heart Rhythm 4/2013 2013;10:604-606.
27. Agac M T, Erkan H, Korkmaz L. Conversion of Brugada type I to type III and successful control of recurrent ventricular arrhythmia with cilostazol. ArchCardiovasc Dis Jan. 16, 2013 2013.
28. Hasegawa K, Ashihara T, Kimura H, Jo H, Itoh H, Yamamoto T, Aizawa Y, Horie M. Long-term pharmacological therapy of Brugada syndrome: is J-wave attenuation a marker of drug efficacy? InternMed 2014 2014;53:1523-1526.
29. Shinohara T, Ebata Y, Ayabe R, Fukui A, Okada N, Yufu K, Nakagawa M, Takahashi N. Combination therapy of cilostazol and bepridil suppresses recurrent ventricular fibrillation related to J-wave syndromes. Heart Rhythm Aug. 1, 2014 2014; 11:1441-1445.
30. Nam G B, Kim Y H, Antzelevitch C. Augmentation of J waves and electrical storms in patients with early repolarization. NEnglJ Med May 8, 2008 2008;358:2078-2079.
31. Haissaguerre M, Chatel S, Sacher F, et al. Ventricular fibrillation with prominent early repolarization associated with a rare variant of KCNJ8/KATP channel. Journal of cardiovascular electrophysiology January 2009;20:93-98.
32. Wilde A A, Postema P G, Di Diego J M, Viskin S, Morita H, Fish J M, Antzelevitch C. The pathophysiological mechanism underlying Brugada syndrome: depolarization versus repolarization. J Mol Cell Cardiol 10/2010 2010;49:543-553.
33. Morita H, Zipes D P, Nishii N, Miura D, Nagase S, Hata Y, Nakamura K, Kusano K F, Ohe T, Wu J, Ito H. Impact of sodium channel dysfuction on arrhythmogenesis in Brugada syndrome. Circulation Nov. 3, 2009 2009;120:S696.

Each and every patent, patent application, and publication, including publications listed herein and publicly available nucleic acid and amino acid sequences cited throughout the disclosure, is expressly incorporated herein by reference in its entirety. While this invention has been disclosed with reference to specific embodiments, it is apparent that other embodiments and variations of this invention are devised by others skilled in the art without departing from the true spirit and scope of the invention. The appended claims include such embodiments and equivalent variations.

1. A method for treating a J Wave Syndrome (JWS) in a patient in need thereof, comprising administering an effective amount of at least one I_{TO} inhibitor or I_{NA} augmentor.

2. The method of claim 1 wherein the JWS is Brugada syndrome (BrS) or early repolarization syndrome (ERS).

3. The method of claim 1, wherein the at least one I_{TO} inhibitor or I_{NA} augmentor is selected from Table 1.

4. The method of claim 1, wherein the at least one I_{TO} inhibitor is Arumenamide-787 (AR-787).

5. The method of claim 1, wherein treatment
a) effectively suppresses the electrocardiogramd/or arrhythmic manifestations of JWS,
b) reduces instances of ventricular fibrillation or sudden cardiac death compared to an untreated control, or
c) reduces the amplitude of J waves when compared to an untreated control.

6-7. (canceled)

8. The method of claim 1, wherein at least one I_{TO} inhibitor and/or I_{NA} augmentor is administered via enteral, parenteral, topical, or pulmonary administration.

9. The method of claim 1, further comprising administering an I_{CA} augmentor, a β-adrenergic agonist, quinidine, bepridil, or denopamine.

10. The method of claim 9, wherein the I_{CA} augmentor is isoproterenol, cilostazol and/or milrinone.

11-12. (canceled)

13. The method of claim 1, wherein the patient has hypothermia.

14. The method of claim 13, wherein the hypothermia is mild hypothermia, moderate hypothermia, severe hypothermia, or therapeutic hypothermia.

15. A method for ameliorating symptoms of a J Wave Syndrome (JWS) phenotype in a patient having hypothermia, comprising administering an effective amount of at least one I_{TO} inhibitor or I_{NA} augmentor.

16. The method of claim 15, wherein the hypothermia is mild hypothermia, moderate hypothermia, severe hypothermia, or therapeutic hypothermia.

17. The method of claim 15, wherein the at least one I_{TO} inhibitor or I_{NA} augmentor is selected from Table 1.

18. The method of claim 15, wherein the at least one I_{TO} inhibitor is Arumenamide-787 (AR-787).

19. The method of claim 15, wherein treatment
a) effectively suppresses the electrocardiographic or arrhythmic manifestations of JWS,
b) reduces instances of ventricular fibrillation or sudden cardiac death compared to an untreated control, or
c) reduces the amplitude of J waves when compared to an untreated control.

20-21. (canceled)

22. The method of claim 15, wherein at least one I_{TO} inhibitor and/or I_{NA} augmentor is administered via enteral, parenteral, topical, or pulmonary administration.

23. The method of claim 15, further comprising administering an I_{CA} augmentor, a one β-adrenergic agonist, quinidine, bepridil, or denopamine.

24. The method of claim 15, wherein the I_{CA} augmentor is isoproterenol, cilostazol and milrinone.

25-26. (canceled)

27. The method of claim 15, further comprising treating the hypothermia.

28. The method of claim 15, wherein treating the hypothermia comprises at least one of wrapping the patient in blankets, administering warm fluids by mouth, immersing the patient in a warm water bath, and direct warming of a patient's blood.
